(19) United States
(12) Patent Application Publication
(10) Pub. No.: US 2026/0216166 A1
(43) Pub. Date: Jul. 30, 2026

(54) COMPOUND FOR TREATMENT OF ERECTILE DYSFUNCTION

(71) Applicant: Initiator Pharma A/S, Copenhagen N (DK)

(72) Inventors: Ulf Simonsen, Copenhagen N (DK); Mikael Thomsen, Copenhagen N (DK)

(21) Appl. No.: 19/144,662

(22) PCT Filed: Jan. 3, 2024
(86) PCT No.: PCT/EP2024/050061

§ 371 Date: Jun. 30, 2025

(30) Foreign Application Priority Data
Jan. 3, 2023 (EP) 23150113.1
Oct. 5, 2023 (EP) 23201933.1

(51) Int. Cl. A61K 31/46; A61K 31/4985; A61K 31/506; A61K 31/519; A61K 31/53; A61P 15/10

(52) U.S. Cl. CPC A61K 31/46; A61K 31/4985; A61K 31/506; A61K 31/519; A61K 31/53; A61P 15/10

(57) ABSTRACT

The present invention relates to compound for treatment of erectile dysfunction.

### TECHNICAL FIELD

The present invention relates to compound for treatment of erectile dysfunction.

### BACKGROUND

Erectile Dysfunction (ED) is defined as the inability to obtain and maintain an erection sufficient for sexual intercourse, is a disorder that affects a high and increasing number of men. ED patients have been found to have decreased quality-of-life (QoL) caused by various psychosocial reasons such as low self-esteem, depression, sadness, anger, frustration, anxiety and relationship problems.

While current treatments, such as phosphodiestearase 5 (PDE5) inhibitors have shown improvements, still approximately 30% to 40% of men with ED do not respond to the PDE5i drug therapy and this group of patients still experience low QoL. Also, patients with erectile dysfunction (ED) with co-morbidities may be resistant to the currently recommended treatments with phosphodiesterase type 5 (PDE5) inhibitors and/or combinations with other oral treatments.

A higher success rate can be obtained by injection therapy with a combination of drugs capable of relaxing penile smooth muscle, but currently, there is no peroral treatment. Accordingly, restoring altered signal transduction and underlying pathophysiological mechanisms in the genital tissue can effectively treat ED. However, comorbid neurological disease, diabetes mellitus, and severe vascular disease may also negatively affect the central pathways modulating erectile function. Moreover, medications for central nervous system disorders may affect function and libido with a predisposition to affect more men than women. Therefore, there is a marked unmet need for new treatment modalities of ED that can address the issues highlighted above.

### SUMMARY

As outlined above, a compound able to offer new treatment modalities for erectile dysfunction is highly desired. The present disclosure provides for a compound useful in the treatment of erectile dysfunction, including diverse types of erectile dysfunction.

Thus, in one main aspect, the present disclosure provides a compound of formula (I)

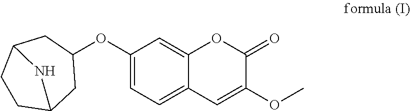 [SMILES: COc1cc2ccc(OC3CC4CCC(C3)N4)cc2oc1=O]

or a pharmaceutically acceptable salt thereof, for use in the treatment, prevention, reduction or alleviation of erectile dysfunction in a subject, wherein the compound is administered in a dose from about 0.001 mg/kg to about 1 mg/kg.

The inventors have shown that the use of a compound of formula I as described herein has activity in treatment of erectile dysfunction. The inventors have found that the compound has effect in different animal models, including humans and that the compound for use as described herein effectively treats signs of erectile dysfunction. In humans, the inventors have found the compound improves duration of erectile events, rigidity and tumescence.

The inventors have surprisingly demonstrated in studies in healthy volunteers that individual doses of the compound of 10 mg or below are linked to a lower incidence of treatment-emergent adverse effects (TEAEs) compared to higher doses. In studies with subjects having erectile dysfunction, it has also been shown that individual doses of 5 mg of IP2015 are effective in treating erectile dysfunction in human subjects compared to placebo.

Another aspect of the present disclosure provides for a solid dosage form comprising a compound of formula I,

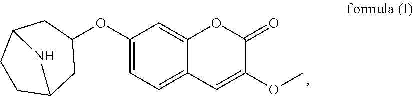 [SMILES: COc1cc2ccc(OC3CC4CCC(C3)N4)cc2oc1=O]

or a pharmaceutically acceptable salt thereof, wherein the compound is present in an amount from about 1 mg to about 10 mg.

Another aspect of the present disclosure provides for composition comprising a compound of formula (I),

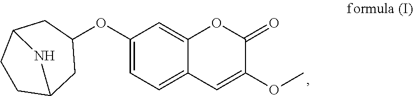 [SMILES: COc1cc2ccc(OC3CC4CCC(C3)N4)cc2oc1=O]

and one or more PDE5 inhibitor(s).

### DESCRIPTION OF DRAWINGS

FIG. 1. Original traces show increased intracavernosal pressure induced by the cavernous nerve's electrical field stimulation (EFS). Administration of A) vehicle does not change the pressure, while (B-D) IP2015 administration induces dose-dependent increases in intracavernous pressure (spontaneous erections, SE) without changing the responses to EFS. E-G) Frequency, duration, and magnitude of spontaneous erections after intravenous infusion of vehicle, IP2015 0.1 mg/kg, 1 mg/kg, or 10 mg/kg. The effect on EFS is reported in the supplementary data file. (H-J) Intravenous infusion of vehicle and IP2015 (1 mg/kg) in diabetic db/db mice. IP2015 increased H) frequency, I) duration and J) the magnitude of spontaneous erectile responses in db/db mice (n=7). The results are means±s.e.mean. *P<0.05 versus vehicle control.

FIG. 2. Effect of IP2015 and synergy with the phosphodiesterase type 5 inhibitor, sildenafil, on erections. Average rises in A) frequency, B) duration, and (C) magnitude of erectile responses induced by IP2015 in the absence (n=6) and the presence (n=5) of the phosphodiesterase type 5 inhibitor, sildenafil (1 mg/kg) in rats. The results are means±s.e.mean. *P<0.05 versus control.

FIG. 3. IP2015 induces relaxations mediated by dopamine D1 receptors and nitric oxide in isolated rat corpus cavernosum strips. A) At baseline tension, increasing IP2015 concentrations induces relaxations, which are inhibited in the presence of the dopamine D₁ receptor antagonist, SCH23390, and unaltered in the presence of the dopamine D₂ receptor antagonist, clozapine (n=6). B) Average relaxations induced by IP2015 in preparations with and without endothelium (n=5). C) Concentration-response curves for IP2015 in the presence of N^{G}-nitro-L-arginine (L-NOARG), guanethidine, and sildenafil at baseline tension (n=8). The results are means±s.e.means. *P<0.05 versus control.

FIG. 4. The study design of a randomised, double-blind, placebo-controlled, sequential group study to investigate ascending single oral doses of IP2015 in healthy male subjects.

FIG. 5. The study design of a randomised, double-blind, placebo-controlled, two-period, crossover study to assess single oral doses of IP2015 in male patients with ED.

FIG. 6. The study design of the stimulus challenge.

FIG. 7. Mean plasma concentrations of IP2015 following single ascending doses (linear scale).

FIG. 8. Mean plasma concentrations of IP2015 following single ascending doses (semi-logarithmic scale).

FIG. 9. Mean plasma concentrations of IP2015 for single dose (linear scale).

FIG. 10. Mean plasma concentrations of IP2015 for single dose (semi-logarithmic scale).

FIG. 11. The response to question Q3 in the international index of erectile function scale 15 (IIEF-15): When you attempted intercourse, how often were you able to penetrate (enter) your partner during the last week?

FIG. 12. The response to question Q4 in the International Index of erectile function scale 15 (IIEF-15): During sexual intercourse, how often were you able to maintain your erection after you had penetrated (entered) your partner?

FIG. 13. The overall response to questions in the International Index of erectile function scale 15 (IIEF-15).

### DEFINITIONS

“Pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

By “IP2015” or pudafensine or “compound I” is meant the compound of formula I. The compound of formula I is:

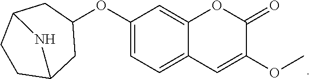 [SMILES: COc1cc2ccc(OC3CC4CCC(C3)N4)cc2oc1=O]

The term “pharmaceutically acceptable salt” of a compound refers to a salt that is pharmaceutically acceptable, as defined herein, and preferably possesses the desired pharmacological activity of the parent compound. Pharmaceutically acceptable salts include acid addition salts formed with inorganic acids; or formed with organic acids; or salts formed when an acidic proton present in the parent compound either is replaced by a metal ion; or coordinates with an organic or inorganic base.

Whenever a chiral carbon is present in a chemical structure, it is intended that all stereoisomers associated with that chiral carbon are encompassed by the structure, unless otherwise specified. Using the Cahn-Ingold-Prelog RS notational system, any asymmetric carbon atom may be present in the (R)- or (S)-configuration, and the compound may be present as a mixture of its stereoisomers, e.g. a racemic mixture, or one stereoisomer only.

The compound of the invention may exist in a tautomeric form. Any such tautomer is considered to be within the scope of the invention.

Also, in the compound of formula I as defined herein, any hydrogen atom may be replaced by a deuterium (2H), and any such deuterated compound of formula I, comprising one or more deuterium atoms in place of the corresponding number of hydrogen atoms, is considered to be within the scope of the invention.

It is known in the art that prodrugs can be produced. The person skilled in the art will know which types of molecular moieties can be introduced on a drug to produce a prodrug. It is considered that prodrugs relating to the compound of formula I are within the scope of the invention.

As used herein the terms “treatment” or “treating” is an approach for obtaining beneficial or desired results including clinical results. Beneficial or desired clinical results can include, but are not limited to, alleviation or amelioration of one or more symptoms or conditions, diminishment of extent of disease or disorder, stabilized (i.e., not worsening) state of disease or disorder, prevention of the disease or disorder, delay or slowing of disease or disorder progression, amelioration or palliation of the disease state, and remission (whether partial or total) whether detectable or undetectable.

As used herein, “erectile dysfunction” refers to a disorder involving the failure of a male mammal to achieve erection, ejaculation, or both. Symptoms of erectile dysfunction include an inability to achieve or maintain an erection, ejaculatory failure, premature ejaculation, or inability to achieve an orgasm. Erectile dysfunction (hereinafter also referred to as “ED”) is also called “impotence”, “erectile functional disorder” or “erectile disorder”. ED is divided into the organic factors (caused by or associated with another medical condition such as arterial sclerosis, nerve damage, diabetes etc.), the psychological factors (caused by psychological stress) and the mixed factor (generated by combining the both elements of the organic factor and the psychological factor) according to causes.

As used herein, “organic” erectile dysfunction refers to erectile dysfunction which is caused by a medical condition that physically impairs the delivery of adequate blood flow to, or prevent maintenance of sufficient blood within, the erectile tissues of the penis. Organic causes of ED can be for example, vascular or neurologic. In organic ED caused by vascular conditions the vascular system responsible for either achieving or maintaining an erection is dysfunctional. Vascular causes of organic ED can be for example, hypertension, diabetes and heart disease that atherosclerosis or congestive heart failure may impose. Any of these conditions can compromise the delivery of blood necessary to achieve an erection. Fibrotic changes in the penile tissue may also difficult the maintaince of adequate blood flow into the erectile penis. Neurological diseases affecting the central or peripheral nervous system are also a cause of organic ED, for example advanced diabetes. Organic ED may also be cause due to surgery, such as prostate, pelvic surgery or other types of surgery or be caused by medication. As much as 90% of ED is accounted for by organic causes. The remaining 10% of ED is considered non-organic, or psychogenic.

As used herein the term “comorbid” is used to refer to disease or pathological conditions that occur simultaneously with another condition in a subject, regardless of the relationship between them.

The international index of erectile function (IIEF-15) questionnaire is a validated, multi-dimensional, self-administered investigation that has been found useful in the clinical assessment of erectile dysfunction and treatment outcomes in clinical trials. A score of 0-5 is awarded to each of the 15 questions that examine the main domains of male sexual function: erectile function, orgasmic function, sexual desire and intercourse satisfaction.

The domains according to specific questions are:
Domain A—Erectile function (Q1,2,3,4,5,15)
Domain B—Orgasmic Function (Q9,10)
Domain C—Sexual Desire (Q11,12)
Domain D—Intercourse Satisfaction (Q6,7,8)
Domain E—Overall Satisfaction (Q13, 14)

The IIEF-5 is a 5-item version of the IIEF-15. The possible scores of IIEF-5 range from 5 to 25, and erectile dysfunction can be classified into 5 categories based on the scores: severe (5-7), moderate (8-11), mild to moderate (12-16), mild (17-21), and no ED (22-25).

As used herein, “formulation” is the result of combining different substances, including the active ingredient, to produce a final product.

The term “about” as used herein to refer to an amount or percentage is to be interpreted as a variation of ±10% with respect the value of the amount or percentage it refers to, such as ±5%.

### DETAILED DESCRIPTION

### Compound for Use

One embodiment of the present disclosure provides for a compound of formula (I),

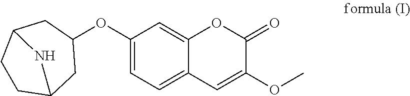 [SMILES: COc1cc2ccc(OC3CC4CCC(C3)N4)cc2oc1=O]

or a pharmaceutically acceptable salt thereof for use in the treatment, prevention, reduction or alleviation of erectile dysfunction in a subject, wherein the compound is administered in a dose from about 0.001 mg/kg to about 1 mg/kg. The compound of the present disclosure is a monoamine reuptake inhibitor. Compounds may be tested for their ability to inhibit reuptake of the monoamines dopamine, noradrenaline and serotonin in synaptosomes e.g. such as described in WO 97/30997 or WO 97/16451.

In one embodiment, the compound of formula (I) has the structure of formula (Ia);

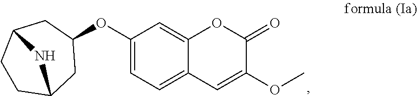 [SMILES: COc1cc2ccc(O[C@@H]3C[C@H]4CC[C@@H](C3)N4)cc2oc1=O]

or a pharmaceutically acceptable salt thereof.

In one embodiment, the compound is 7-[(8-azabicyclo[3.2.1]octan-3-yl)oxy]-3-methoxy-chromen-2-one, or a pharmaceutically acceptable salt thereof. In one embodiment, the compound is exo-7-[(8-azabicyclo[3.2.1]octan-3-yl)oxy]-3-methoxy-chromen-2-one, or a pharmaceutically acceptable salt thereof. In one embodiment, the name “IP2015” means the compound of formula I. In one embodiment, the name “IP2015” means the compound of formula Ia. In a specific embodiment of the present disclosure “IP2015” means the hydrochloride of the compound of formula I.

In one embodiment of the disclosure, the compound is exo-7-[(8-azabicyclo[3.2.1]octan-3-yl)oxy]-3-methoxy-chromen-2-one hydrochloride.

One embodiment of the disclosure provides for a compound of formula I,

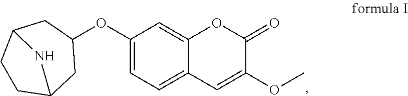 [SMILES: COc1cc2ccc(OC3CC4CCC(C3)N4)cc2oc1=O]

or a pharmaceutically acceptable salt thereof, for use in the treatment, prevention, reduction or alleviation of erectile dysfunction in a subject as described herein.

In some instances, the erectile dysfunction is organic erectile dysfunction, such as erectile dysfunction associated with or due to metabolic syndrome, such as erectile dysfunction associated with or due to diabetes, such as erectile dysfunction associated with or due to a vascular disease, such as a cardiovascular disease, or erectile dysfunction associated with or due to neurological damage.

Alternatively, the erectile dysfunction can be, for example, associated with or due to medical condition, such as injury, the effects of surgery or radiation treatment, or it can be associated with or due to psychological or behavioural factors, including mental disorders, or use of medication or use of a psychoactive substance.

One embodiment of the disclosure provides for a compound of formula I,

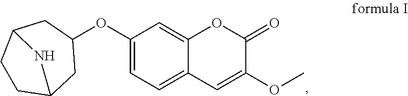 [SMILES: COc1cc2ccc(OC3CC4CCC(C3)N4)cc2oc1=O]

or a pharmaceutically acceptable salt thereof, for use in the treatment, prevention, reduction or alleviation erectile dysfunction in a subject, wherein the compound is administered in a dose from about 0.001 mg/kg to about 1 mg/kg.

In one embodiment, the compound of formula I is administered in an amount per individual dose from about 0.001 mg/kg to about 1 mg/kg, such as 0.005 mg/kg, such as 0.01 mg/kg, such as 0.015 mg/kg, such as 0.02 mg/kg, such as 0.025 mg/kg, such as 0.03 mg/kg, such as 0.035 mg/kg, such as 0.04 mg/kg, such as 0.045 mg/kg, such as 0.05 mg/kg, such as 0.055 mg/kg, such as 0.06 mg/kg, such as 0.065 mg/kg, such as 0.07 mg/kg, such as 0.075 mg/kg, such as 0.08 mg/kg, such as 0.085 mg/kg, such as 0.09 mg/kg, such as 0.095 mg/kg, such as 0.1 mg/kg, such as 0.15 mg/kg, such as 0.2 mg/kg, such as 0.25 mg/kg, such as 0.3 mg/kg, such as 0.35 mg/kg, such as 0.4 mg/kg, such as 0.45 mg/kg, such as 0.5 mg/kg, such as 0.55 mg/kg, such as 0.6 mg/kg, such as 0.65 mg/kg, such as 0.7 mg/kg, such as 0.75 mg/kg, such as 0.8 mg/kg, such as 0.85 mg/kg, such as 0.9 mg/kg, such as 0.95 mg/kg, such as 1 mg/kg per individual dose.

In one embodiment, the compound is administered in an amount from about 0.05 mg/kg to about 1 mg/kg per individual dose.

In one embodiment, the compound is administered orally in an amount per individual dose from about 0.01 mg/kg to about 1 mg/kg, such as 0.01 mg/kg, such as 0.05 mg/kg, such as 0.1 mg/kg, such as 0.15 mg/kg, such as 0.2 mg/kg, such as 0.25 mg/kg, such as 0.3 mg/kg, such as 0.35 mg/kg, such as 0.4 mg/kg, such as 0.45 mg/kg, such as 0.5 mg/kg, such as 0.55 mg/kg, such as 0.6 mg/kg, such as 0.65 mg/kg, such as 0.7 mg/kg, such as 0.75 mg/kg, such as 0.8 mg/kg, such as 0.85 mg/kg, such as 0.9 mg/kg, such as 0.95 mg/kg, such as 1 mg/kg per individual dose.

In one embodiment, the compound is administered intravenously in an amount per individual dose from about 0.001 to about 0.1 mg/kg, such as 0.001 mg/kg, such as 0.005 mg/kg, such as 0.01 mg/kg, such as 0.015 mg/kg, such as 0.02 mg/kg, such as 0.025 mg/kg, such as 0.03 mg/kg, such as 0.035 mg/kg, such as 0.04 mg/kg, such as 0.045 mg/kg, such as 0.05 mg/kg, such as 0.055 mg/kg, such as 0.06 mg/kg, such as 0.065 mg/kg, such as 0.07 mg/kg, such as 0.075 mg/kg, such as 0.08 mg/kg, such as 0.085 mg/kg, such as 0.09 mg/kg, such as 0.095 mg/kg, such as 0.1 mg/kg per individual dose.

In one embodiment, the compound is preferably administered orally in an amount per individual dose from about 0.035 mg/kg to 0.135 mg/kg, such as from 0.040 mg/kg to 0.110 mg/kg, such as from 0.042 mg/kg to 0.100 mg/kg, such as from 0.0450 mg/kg to 0.100 mg/kg, such as from 0.045 mg/kg to 0.091 mg/kg, such as from 0.050 mg/kg to 0.091 mg/kg.

In one embodiment, the compound is administered orally in an amount per individual dose from about 0.050 mg/kg to 0.053 mg/kg, or 0.053 mg/kg to 0.056 mg/kg, or 0.056 mg/kg to 0.059 mg/kg, or 0.059 mg/kg to 0.063 mg/kg, or 0.063 mg/kg to 0.067 mg/kg, or 0.067 mg/kg to 0.071 mg/kg, or 0.071 mg/kg to 0.077 mg/kg, or 0.077 mg/kg to 0.083 mg/kg, or 0.083 mg/kg to 0.091 mg/kg.

In one embodiment, the compound is administered orally in an amount per individual dose from 0.075 mg/kg to 0.250 mg/kg, such as from 0.080 mg/kg to 0.222 mg/kg, such as from 0.083 mg/kg to 0.200 mg/kg, such as from 0.091 mg/kg to 0.185 mg/kg, such as from 0.100 mg/kg to 0.185 mg/kg, such as from 0.050 mg/kg to 0.185 mg/kg.

One embodiment of the disclosure provides for a compound of formula I,

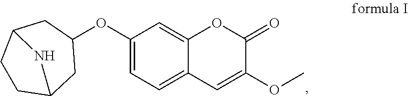 [SMILES: COc1cc2ccc(OC3CC4CCC(C3)N4)cc2oc1=O]

or a pharmaceutically acceptable salt thereof, for use in the treatment, prevention, reduction or alleviation of pain in a subject, wherein the compound is administered in a dose from about 0.5 mg to about 20 mg per individual dose, such as from about 1 mg to about 15 mg per individual dose, such as from about 5 mg to about 10 mg per individual dose, such as about 5 mg per individual dose, such as about 10 mg per individual dose.

In one embodiment, the compound is administered in an amount from about 0.5 mg to 20 mg per individual dose, such as about 0.5 mg, such as about 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg or 20 mg per individual dose.

In the present disclosure, it is shown for the first time in healthy volunteers that doses of 10 mg or below are associated with lower side effects than doses of 16.2 mg. Thus, in one embodiment, the compound is administered in an amount from about 1 mg to 10 mg per individual dose, such as from about 3 mg to about 10 mg, such as about 5 mg or such as about 10 mg.

In one embodiment, the compound is administered in an amount from about 1 mg to about 10 mg per individual dose as described herein and said administration demonstrates adverse events in fewer than 85 subjects per 100 subjects, such as demonstrates adverse events in fewer than 84, 83, 82, 81, or 80 subjects per 100 subjects.

In one embodiment, the compound is administered in an amount of about 5 mg per individual dose as described herein and said administration demonstrates adverse events in fewer than 80 subjects per 100 subjects, such as demonstrates adverse events in fewer than 79, 78, 77, or 76 subjects per 100 subjects.

In one embodiment, the compound is administered in an amount from about 1 mg to about 10 mg per individual dose as described herein and said administration demonstrates moderate adverse events in fewer than 23 subjects per 100 subjects, such as demonstrates moderate adverse events in fewer than 22, 21, 20, 19, 18, or 16 subjects per 100 subjects.

In one embodiment, the compound is administered in an amount of about 5 mg per individual dose as described herein and said administration demonstrates moderate adverse events in fewer than 13 subjects per 100 subjects, such as demonstrates moderate adverse events in fewer than 12, 11, 10, 9, or 8 subjects per 100 subjects.

In one embodiment, the compound is administered in an amount from about 1 mg to about 10 mg per individual dose as described herein and said administration demonstrates mild adverse events in fewer than 82 subjects per 100 subjects, such as demonstrates mild adverse events in fewer than 81, 80, 79, 78, or 77 subjects per 100 subjects.

In one embodiment, the compound is administered in an amount of about 5 mg per individual dose as described herein and said administration demonstrates mild adverse events in fewer than 75 subjects per 100 subjects, such as demonstrates mild adverse events in fewer than 74, 73, 72, 71 or 70 subjects per 100 subjects.

In one embodiment, the compound is administered in an amount of about 10 mg or less per individual dose as described herein and said administration demonstrates fewer than 15 total moderate adverse events per every 40 to 50 subjects, such as fewer than 12 total moderate adverse events per every 40 to 45 subjects, such as fewer than 10 total moderate adverse events per every 43 subjects.

In one embodiment, the compound is administered in an amount of about 5 mg or less per individual dose as described herein and said administration demonstrates fewer than 10 total moderate adverse events per every 40 to 50 subjects, such as fewer than 7 total moderate adverse events per every 40 to 45 subjects, such as fewer than 6 moderate adverse events per every 42 subjects.

In one embodiment, the compound is administered in an amount of about 10 mg or less per individual dose as described herein and said administration demonstrates fewer than 40 total mild adverse events per every 40 to 50 subjects, such as fewer than 37 total mild adverse events per every 40 to 45 subjects, such as fewer than 34 total mild adverse events per every 43 subjects.

In one embodiment, the compound is administered in an amount of about 5 mg or less per individual dose as described herein and said administration demonstrates fewer than 35 total mild adverse events per every 40 to 50 subjects, such as fewer than 32 total mild adverse events per every 40 to 45 subjects, such as fewer than 30 mild adverse events per every 42 subjects.

In one embodiment, the compound is administered in an amount from about 1 mg to about 10 mg per individual dose as described herein and said administration demonstrates treatment-related adverse events in fewer than 58 subjects per 100 subjects, such as demonstrates treatment-related adverse events in fewer than 57, 56, 55, or 54 subjects per 100 subjects.

In one embodiment, the compound is administered in an amount of about 5 mg per individual dose as described herein and said administration demonstrates treatment-related adverse events in fewer than 28 subjects per 100 subjects, such as demonstrates treatment-related adverse events in fewer than 27, 26, 25, or 24 subjects per 100 subjects.

Mild adverse events refer to adverse events where the subject shows transient or mild discomfort with no limitation in activity and not requiring medical intervention. Moderate adverse events relate to adverse events where the subject shows mild to moderate discomfort, which may cause mild limitation in activity (not always) and may require some assistance.

Treatment-related adverse events are adverse events which could either be possibly or probably related to the treatment.

In one embodiment, the compound is administered daily. In one embodiment, the compound is administered more than once a day, such as 2 times a day, such as 3 times a day, such as 4 times a day.

In one embodiment, the compound is administered once a week. In one embodiment, the compound is administered twice a week.

In one embodiment, the total daily dose of the compound is from about 0.5 mg to about 100 mg, such as 0.6, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 mg.

In one embodiment, the daily dose of the compound is from about 1 to about 10 mg, such as from about 2 mg to about 10 mg, such as from about 3 mg to about 10 mg, such as from about 4 mg to about 10 mg, such as about 5 mg or such as about 10 mg total daily dose.

In the present disclosure, it is for the first time shown that individual doses of 5 mg of the compound of formula I are effective in the treatment of erectile dysfunction in subjects. Individual doses of 5 mg of IP2015 produced significant improvements in I compared to placebo. Thus, in one embodiment, the compound of formula I is administered to the subject at about 5 mg per individual dose. In one embodiment, the compound of formula I is administered to the subject at 5 mg per individual dose.

In one embodiment, the compound of formula I is administered to the subject at 5 mg per individual dose, wherein the subject has a weight from about 55 kg to about 130 kg. In one embodiment, the compound of formula I is administered to the subject at 5 mg per individual dose, wherein the subject was a weight from about 60 kg to about 115 kg. In one embodiment, the compound of formula I is administered to the subject at 0.041 mg/kg to about 0.083 mg/kg per individual dose.

In one embodiment, the compound for use is for the treatment, prevention or alleviation of erectile dysfunction, wherein the erectile dysfunction is:
(i) Associated with, or due to, a medical condition, such as injury, the effects of surgery or radiation treatment; and/or
(ii) Associated with, or due to, psychological or behavioural factors, including mental disorders; and/or
(iii) Associated with, or due to, use of medication or use of a psychoactive substance.

In one embodiment, the compound for use is for the treatment, prevention or alleviation of erectile dysfunction, which is organic erectile dysfunction. Organic erectile dysfunction refers to erectile dysfunction which is caused by a medical condition that physically impair the delivery of adequate blood flow to, or prevent maintenance of sufficient blood within, the erectile tissues of the penis. Organic causes of ED can be for example, vascular or neurologic.

In one embodiment, the compound for use is for the erectile dysfunction which is lifelong erectile dysfunction or acquired erectile dysfunction. Lifelong erectile dysfunction is erectile dysfunction, which the subject has always experienced. Acquired erectile dysfunction is erectile dysfunction, which follows a period of time during which the person did no experience erectile dysfunction. In one embodiment, the compound for use is for the erectile dysfunction which is generalised erectile dysfunction or situational erectile dysfunction. Generalised erectile dysfunction is characterised by absent, inadequate or absent erectile response or erectile function in all circumstances and sexual stimulus. Situational erectile dysfunction is characterised by absent, inadequate or absent erectile response or erectile function in some circumstances, with some partners, or in response to some stimuli but not in other situations.

In one embodiment, the erectile dysfunction is comorbid in the subject with one or more condition(s) selected from the group consisting of:
(i) diabetes,
(ii) metabolic syndrome,
(iii) a vascular disease, such as a cardiovascular disease, and
(iv) a neurological condition.

In one embodiment, the erectile dysfunction is comorbid in the subject with diabetes. In one embodiment, the erectile dysfunction is comorbid in the subject with metabolic syndrome. In one embodiment, the erectile dysfunction is comorbid in the subject with a vascular disease, such as a cardiovascular disease. In one embodiment, the erectile dysfunction is comorbid in the subject with a neurological condition.

In one embodiment, the compound for use is for the erectile dysfunction which is associated with or due to metabolic syndrome.

In one embodiment, the compound for use is for the erectile dysfunction which is associated with or due to diabetes.

In one embodiment, the compound for use is for the erectile dysfunction which is associated with or due to a vascular disease, such as a cardiovascular disease.

In one embodiment the compound for use is for the erectile dysfunction which is associated with or due to neurological damage.

In one embodiment, the compound for use is for the erectile dysfunction which is treatment-emergent erectile dysfunction associated with use of medication. In one embodiment the treatment-emergent erectile dysfunction is an adverse effect originating from treatment of a medicament. In a further embodiment the medicament is selected from the group consisting of antidepressants, NSAIDs, finasteride, antiepileptics and neuroleptics.

In one embodiment, the compound for use is for the erectile dysfunction which is treatment-emergent erectile dysfunction caused by the treatment by and antidepressant medicament.

The compound for use according to the present disclosure may be combined with one or more further therapeutic agent(s). Accordingly, in one embodiment, the subject is administered with a further therapeutic agent effective for the treatment of erectile dysfunction.

In one embodiment, the further therapeutic agent effective for the treatment of erectile dysfunction is a phosphodiestearase-5 inhibitor, such as sildenafil, tadalafil, vardenafil or avanafil; alprostadil. In one embodiment, the further therapeutic agent effective for the treatment of erectile dysfunction is testosterone. In one embodiment, the compound for use is as described herein is combined with cell therapy. In one embodiment, the compound for use as described herein is combined with extracorporal shock therapy.

In one embodiment, the subject is under another treatment for erectile dysfunction. In one embodiment, the subject is under treatment with one or more phosphodiestearase-5 (PDE5) inhibitor(s), such as sildenafil, tadalafil, vardenafil or avanafil.

In one embodiment, the subject is a non-responder to treatment for erectile dysfunction with one or more PDE5 inhibitors. By term “non-responder” it is to be understood that the subject does not respond in the expected way to therapy. Non-responders do not respond as expected to currently established and marketed therapeutic products. For example, a non-responder to erectile dysfunction treatment with PDE5 inhibitors responds sub-optimally to the treatment with PDE5, for example, wherein there is absent or insufficient improvement of erectile dysfunction in response to treatment with a PDE5 inhibitor.

In one embodiment, the compound for use as described herein is able to produce a central effect initiating erection and/or a peripheral effect potentiating erection.

In one embodiment, the compound for use as described herein is able to initiate and/or potentiate erection through smooth muscle relaxation.

In one embodiment, the compound for use as described herein is able to initiate erection by increasing central dopamine. In one embodiment, the compound for use as described herein is able to potentiate erection through nitric oxide release. In one embodiment, the compound for use as described herein is able to initiate erection by increasing central dopamine and a peripheral effect potentiating erection through nitric oxide release.

In one embodiment, the compound is able to increase the number and/or duration and/or frequency of erectile events, penile tumescence and/or the penile rigidity. Tumescence refers to the quality of being or becoming swollen.

In one embodiment, the compound is able to increase the number and/or duration of erectile events, penile tumescence and/or the penile rigidity in the subject during sexual stimulation.

The number and duration of erectile events, penile tumescence and tumescence events and penile rigidity can be measure by methods known in the art such as the Rigiscan device and analysed by the Rigiscan Plus Software¹.

In one embodiment, the compound is able to increase one or more of the parameters obtained with Rigiscan assessment such as duration of an erectile event, average rigidity of an erectile event, time rigid 80-100%, the tip Rigidity Activity Units (RAU) and the base Tumescence activity units (TAU).

In one embodiment, the subject is a mammal. In one embodiment, the subject is a human. Preferably, the subject is a male, such as a human male. In some embodiments, the subject is an adult male. In some embodiments, the subject is a male over the age of 20, such as an adult male over the age of 25, such as an adult male over the age of 30, such as an adult male over the age of 35, such as an adult male over the age of 40, such as an adult male over the age of 45, such as an adult male over the age of 50, such as an adult male over the age of 55, such as an adult male over the age of 60, such as an adult male over the age of 65, such as an adult male over the age of 70, such as an adult male over the age of 75.

In one embodiment, the compound is for use as described herein in a subject, who prior to treatment had an international index of Erectile Function (IIEF-5) of less than 22, such as between 21 and 17, such as between 12 and 16, such as between 8 and 11, such as between 5 and 7.

In one embodiment, the compound is for use in a subject, who prior to treatment had an international index of Erectile Function (IIEF-5) of less than 22, such as 21, such as 20, such as 19, such as 18, such as 17, such as 16, such as 15, such as 14, such as 13, such as 12, such as 11, such as 10, such as 9, such as 8, such as 7, such as 6, such as 5.

In one embodiment, the subject had an international index of erectile function 5 (IIEF-5) of less than 17. In one embodiment, the subject had an international index of erectile function 5 (IIEF-5) of less than 12. In one embodiment, the subject had an international index of erectile function 5 (IIEF-5) of less than 8.

In one embodiment, the compound for use is able to increase the International Index of Erectile Function in the subject. In one embodiment the compound for use is able to increase the IIEF-5 score in the subject by an amount between 1 and 20. In one embodiment the compound for use is able to increase the IIEF-5 score in the subject by an amount between 1 and 20, such as by 1, such as by 2, such as by 3, such as by 4, such as by 5, such as by 6, such as by 7, such as by 8, such as by 9, such as by 10, such as by 11, such as by 12, such as by 13, such as by 14, such as by 15, such as by 16, such as by 17, such as by 18, such as by 19, such as by 20. In one embodiment, the compound for use as described herein is able to increase the IIEF-5 score in the subject by an amount of at least 1, such as by at least 2, such as by at least 3, such as by at least 4, such as by at least 5, such as by at least 6, such as by at least 7, such as by at least 8, such as by at least 9, such as by at least 10.

In one embodiment, the compound for use as describe herein is able to increase the score in one or more questions of the International Index of Erectile Function 15 (IIEF-15) in the subject. In one embodiment, the compound for use as described herein is able to increase the score in one or more questions of Domain A of the IIEF-15, for example an increase in the score of any of questions 1, 2, 3, 4, 5, or 15 of IIEF-15.

In one embodiment, the compound for use as described herein is able to improve one or more selected from: erectile function, orgasmic function, sexual desire, intercourse satisfaction and overall satisfaction as measured by the IIEF-15 questionnaire in its different domains.

In one embodiment, the compound for use is administered by oral administration.

In one embodiment, the compound for use is administered by parenteral administration, such as cutaneous, mucosal, subcutaneous, intramuscular, intraperitoneal, intravenous or intra-arterial injection.

In one embodiment, the compound is formulated in a pharmaceutical composition further comprising a pharmaceutically acceptable diluent, carrier and/or excipient.

In one embodiment, the compound is formulated as a solid dosage form, such as a tablet, a capsule, a pill, granules or a powder.

In one embodiment, the compound according to formula (I) and the one or more further therapeutic agent(s) as described herein are administered in the same formulation.

One aspect of the present disclosure provides for a solid dosage form comprising a compound of formula I,

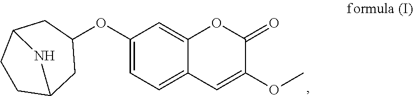 [SMILES: COc1cc2ccc(OC3CC4CCC(C3)N4)cc2oc1=O]

or a pharmaceutically acceptable salt thereof, wherein the compound is present in an amount from about 1 mg to about 10 mg.

One aspect of the present disclosure provides for a composition comprising a compound of formula (I),

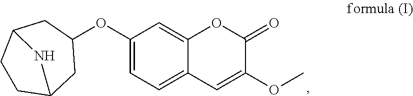 [SMILES: COc1cc2ccc(OC3CC4CCC(C3)N4)cc2oc1=O]

and one or more PDE5 inhibitor(s). In one embodiment, the one or more PDE5 inhibitor(s) are selected from the group consisting of: sildenafil, tadalafil, vardenafil and avanafil. In one embodiment, the compound of formula (I) is present in an amount from about 0.5 mg to about 10 mg.

The inventors have shown that administration of a compound of formula I as described herein is useful for the treatment of erectile dysfunction. Thus, the present disclosure provides for the use of a compound of formula I as described in the section “Compounds for use”.

One embodiment of the present disclosure provides for a use of a compound of formula (I) or a pharmaceutically acceptable salt thereof, for the manufacture of a medicament for the treatment, prevention, reduction or alleviation of erectile dysfunction, wherein said compound is administered in a dose from about 0.001 mg/kg to 1 mg/kg. One embodiment of the present disclosure provides for a use of a compound of formula (I) or a pharmaceutically acceptable salt thereof, for the manufacture of a medicament for the treatment of erectile dysfunction.

One embodiment of the present disclosure provides for a method of treatment, prevention, reduction or alleviation of erectile dysfunction in a subject in need thereof, said method comprising administering to the subject an amount from about 0.001 mg/kg to about 1 mg/kg of a compound of formula (I) or a pharmaceutically acceptable salt thereof.

One embodiment of the present disclosure provides for a method of increasing frequency of erectile responses in a subject said method comprising administering to the subject an amount from about 0.001 mg/kg to about 1 mg/kg of a compound of formula (I) or a pharmaceutically acceptable salt thereof.

One embodiment of the present disclosure provides for a method of increasing duration of erectile responses in a subject said method comprising administering to the subject an amount from about 0.001 mg/kg to about 1 mg/kg of a compound of formula (I) or a pharmaceutically acceptable salt thereof.

One embodiment of the present disclosure provides for a method of increasing the magnitude of erectile responses in a subject said method comprising administering to the subject an amount from about 0.001 mg/kg to about 1 mg/kg of a compound of formula (I) or a pharmaceutically acceptable salt thereof.

One embodiment of the present disclosure provides for a method of achieving a mean plasma concentration of IP2015 from about 1 ng/mL to 50 ng/ml in a subject said method comprising administering to the subject an amount from about 0.001 mg/kg to about 1 mg/kg of a compound of formula (I) or a pharmaceutically acceptable salt thereof.

### Items

1. A compound of formula (I),

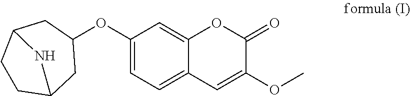 [SMILES: COc1cc2ccc(OC3CC4CCC(C3)N4)cc2oc1=O]

or a pharmaceutically acceptable salt thereof, for use in the treatment, prevention, reduction, or alleviation of erectile dysfunction in a subject, wherein the compound is administered in a dose from about 0.001 mg/kg to about 1 mg/kg.

2. The compound for use according to any one of items 1 and 2, wherein the compound is exo-7-[(8-azabicyclo[3.2.1]octan-3-yl)oxy]-3-methoxy-chromen-2-one or a pharmaceutically acceptable salt thereof.

3. The compound for use according to any one of items 1 to 2, wherein the compound is administered in a dose from about 0.5 mg to about 10 mg.

4. The compound for use according to any one of the preceding items, wherein the subject prior to treatment had an international index of Erectile Function 5 (IIEF-5) of less than 17.

5. The compound for use according to any one of the preceding items, wherein the subject prior to treatment had an international index of Erectile Function 5 (IIEF-5) of less than 12.

6. The compound for use according to any one of the preceding items, wherein the subject is a non-responder of treatment for erectile dysfunction with one or more PDE5 inhibitors.

7. The compound for use according to any one of the preceding items, wherein the erectile dysfunction is organic erectile dysfunction.

8. The compound for use according to any one of the preceding items, wherein the erectile dysfunction is comorbid in the subject with one or more condition(s) selected from the group consisting of:
(i) diabetes,
(ii) metabolic syndrome
(iii) a vascular disease, such as a cardiovascular disease, and
(iv) a neurological condition.

9. The compound for use according to any one of items 1 to 8, wherein the erectile dysfunction is associated with, or due to, diabetes or metabolic syndrome.

10. The compound for use according to any one of items 1 to 8, wherein the erectile dysfunction is associated with, or due to, a vascular disease, such as a cardiovascular disease.

11. The compound for use according to any one of items 1 to 8, wherein the erectile dysfunction is associated with, or due to, a neurological condition.

12. The compound for use according to any one of the preceding items, wherein the compound is able to increase the number and/or duration of erectile events, penile tumescence and/or the penile rigidity.

13. The compound for use according to any one of the preceding items, wherein the compound is able to improve one or more selected from: erectile function, orgasmic function, sexual desire, intercourse satisfaction and overall satisfaction.

14. The compound for use according to any one of the preceding items, wherein the compound is able to increase the score in one or more questions of the International Index of Erectile Function 15 (IIEF-15) in the subject.

15. The compound for use according to any one of the preceding items, wherein the compound is formulated as a solid dosage form, such as a tablet.

### EXAMPLES

### Example 1. Effects of IP2015 on Erectile Function in Rats and Diabetic Mice

The study investigated whether IP2015 can improve erectile function.

### Materials and Methods

For in vivo erectile physiology studies, rats were anaesthetized with pentobarbital sodium (Sygehus Apoteket, Aarhus, Denmark; 50 mg/kg) given intraperitoneally. During the experiment, the rats breathed spontaneously; the body temperature was monitored continuously and was maintained at 37° C. With a midline incision in the perineum, the base of the penis, enclosed by striated muscles, was exposed. The ischiocavernous muscle covering the crus corpus cavernosum was divided on one side, and entrance to the underlying tunica albuginea was given. A 25-gauge needle attached to a heparinized (100 IE ml-1) polyethylene catheter was inserted into the crus corpus cavernosum to measure intracavernous pressure (ICP). A heparinized polyethylene catheter (PE 50) was introduced into the carotid artery to measure mean arterial pressure (MAP). Continuous direct measurements of MAP and ICP were performed with transducers (Disposable BP Transducer, ADInstruments, UK), and registered and analysed on a computerized data acquisition system (PowerLab, ADInstruments).

A stabilizing period of 20-30 min was allowed before registration of basal ICP and MAP. Through a lower abdominal incision, the cavernous nerve was isolated at the lateral aspect of the prostate, and electrical stimulation was performed with a slender bipolar platinum electrode, which was connected to a S48 stimulator (Grass Instrument Co., Boston, MA, U.S.A.). To measure the maximum amplitude of the erectile response, a first stimulation of the cavernous nerve (square wave pulses of 6 Volts, 10 Hz, 1 ms pulse duration for 30 s) was performed.

IP2015, sildenafil, clozapine or vehicle were administered by injection in the jugular vein in volumes of maximum 200 μl. IP2015 was injected at doses of 0.1 and 1 mg/kg intravenously. The observation period of spontaneous erection after injection of IP2015 was 30 min. Clozapine (1 mg/kg) or sildenafil (1 mg/kg) were administered 30 or 10 min prior to 1 mg/kg IP2015 injection, respectively. One group was injected with vehicle alone.

To investigate the involvement of proximal neuronal pathways in the effect of IP2015, mechanical denervation was performed. For mechanical denervation the isolated cavernous nerve was cut distal to the major pelvic ganglion. Absence of erectile response to electrical stimulation verified the efficacy of mechanical denervation.

For functional studies in corpus cavernosum strips, the penis was removed by cutting the crura corpora cavernosa at the point of adhesion to the lower pubic bone, and the corpora cavernosa were then dissected free. The penis was submerged immediately in ice-cold (4° C.) PSS. The tunica albuginea was carefully opened from its proximal extremity of the corpus cavernosum towards the penile shaft and the erectile tissue within the corpus cavernosum was microsurgically dissected free. Change in isometric tension of corpus cavernosum strips (0.5×0.5×3 mm) were investigated in a tissue organ bath system (750TOBS, Danish Myotechnology, Aarhus, Denmark). Silk ligatures were applied at both ends of the strip preparations, which were then suspended between two L-formed metal prongs in thermostatically controlled organ baths (5 ml, 37° C.) containing PSS aerated with a mixture of 5% CO₂ in air (pH 7.4). The bath fluid was routinely changed every 20 min and replaced with fresh PSS, also kept at 37° C. During an equilibration period of 60 min, tension was adjusted until a mean stable tension of 1.2 mN was obtained, as described earlier.

To test the contractility of the preparations, they were exposed to a potassium physiological saline solution (KPSS) of 125 mM and phenylephrine (10-6 M), after each contraction acetylcholine was administrated to check endothelium-dependent relaxations.

To investigate the effect of IP2015 on corpus cavernosum strips, the drug (10 9-3×10-4 M) was administered either in preparations at baseline tension or contracted with phenylephrine (10-6 M) in the absence or presence of L-NOARG (10 4 M), a NO synthase inhibitor, sildenafil (10-7 M), a PDE5 inhibitor, a dopamine D1 receptor antagonist, SCH23390, and a dopamine D2 receptor antagonist, clozapine (10-6 M). The effect of vehicle was also investigated.

### Results

### IP2015 Induces Spontaneous Erections in Rats and Mice.

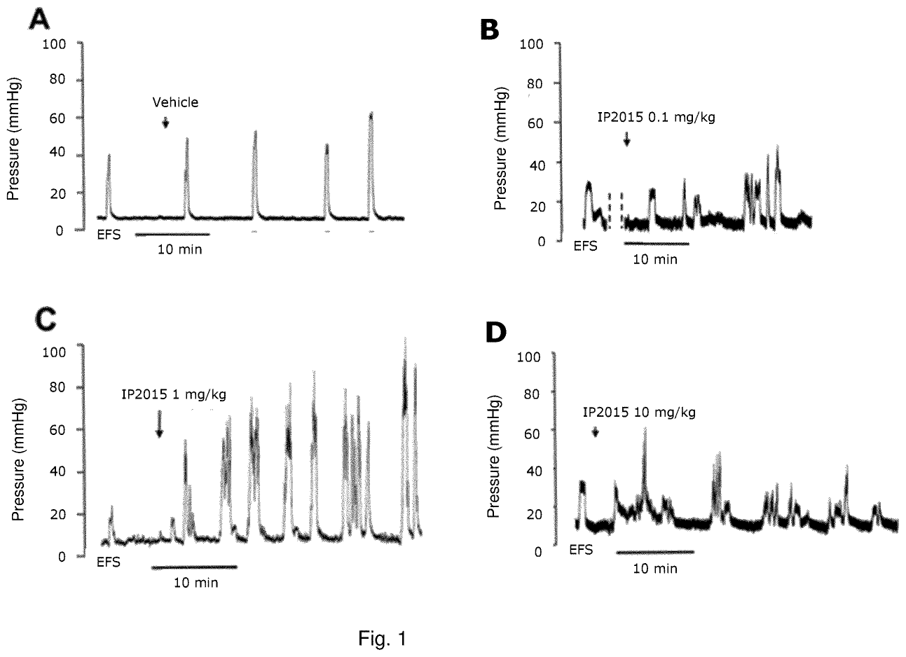

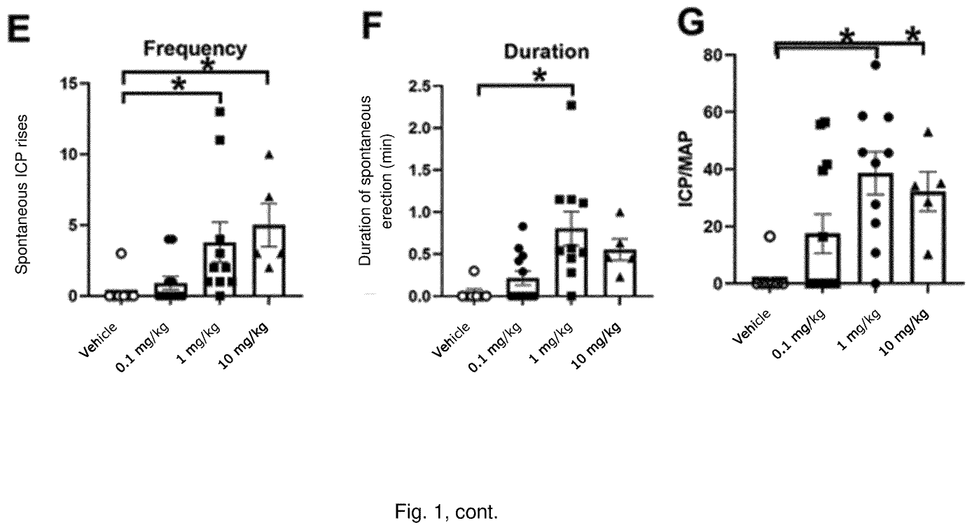

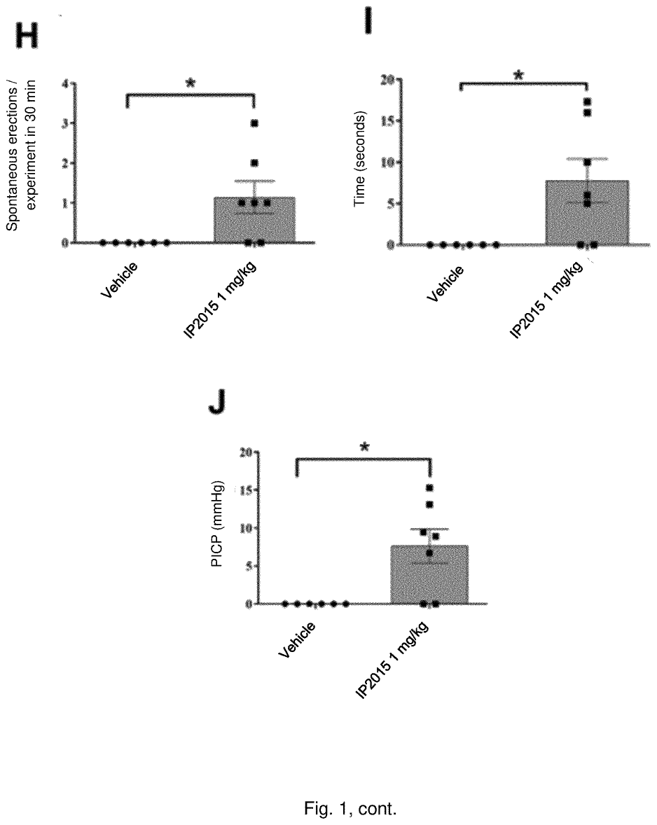

A mean basal intracavernous pressure of 10.2±1.0 mmHg (n=24) and a mean arterial blood pressure of 117.12±3.2 mmHg (n=24) were recorded at the beginning of the experiments. The maximal amplitude of erection evoked by electrical stimulation of the cavernous nerve at the beginning of the experiments was 71.2±1.5 mmHg (n=24). In contrast to the vehicle infusion (FIG. 1A), administration in the jugular vein of 0.1, 1, and 10 mg/kg IP2015 induced transient increases in intracavernosal pressure corresponding to erections (FIG. 1B-D). The frequency, duration, and magnitude of these responses were increased dose-dependently (FIG. 1E-G). The magnitude of erectile responses were characterized by measuring intracavernosal pressure (ICP) increases expressed as a percentage of mean arterial pressure (MAP) and showed they were significantly increased compared to vehicle (FIG. 1G).

To investigate the effects of IP2015 in a simple model of erectile dysfunction, before dissecting its underlying pharmacology in vivo, we assessed its effects in a type-2 diabetic mouse model. Type 2 diabetic db/db mice have decreased erectile function compared to normal C57BL/6 mice and heterozygous db/+ control mice. Infusion of IP2015 in diabetic db/db mice significantly increased the frequency, duration, and magnitude of erectile responses (FIGS. 1H-J).

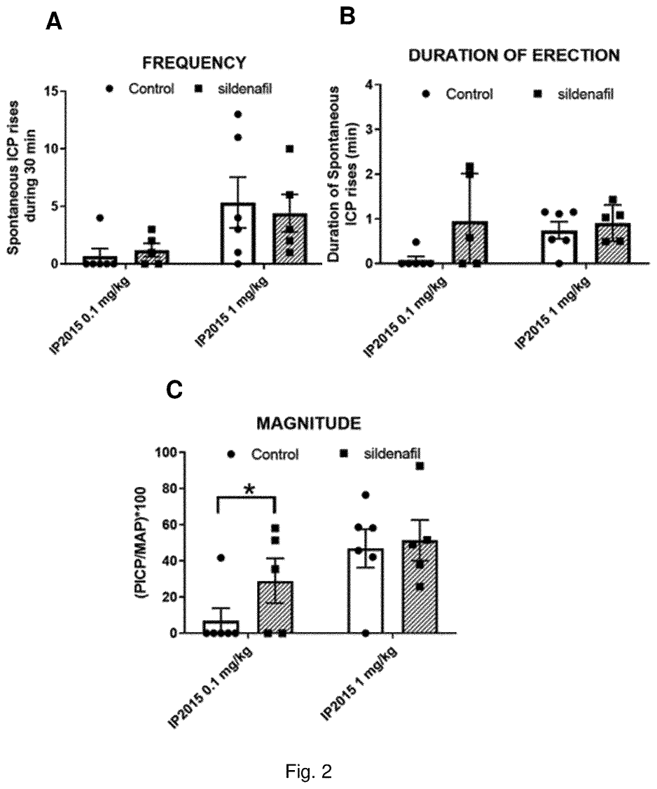

The phosphodiesterase inhibitor sildenafil improves erection by facilitating erectile responses. To investigate the effect of sildenafil on IP2015 administration, IP2015 was infused and induced erectile responses, and treatment with sildenafil markedly increased the duration of these responses (FIG. 2). There was also an effect on the magnitude of erectile responses induced by IP2015 (0.1 mg/kg). These findings suggest that sildenafil potentiates the effect on the erection of low doses of IP2015.

### Effect of IP2015 on Isolated Rat Erectile Tissue

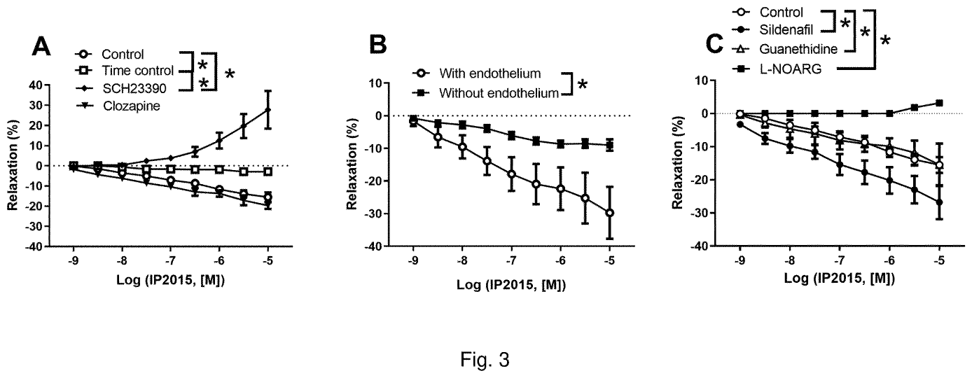

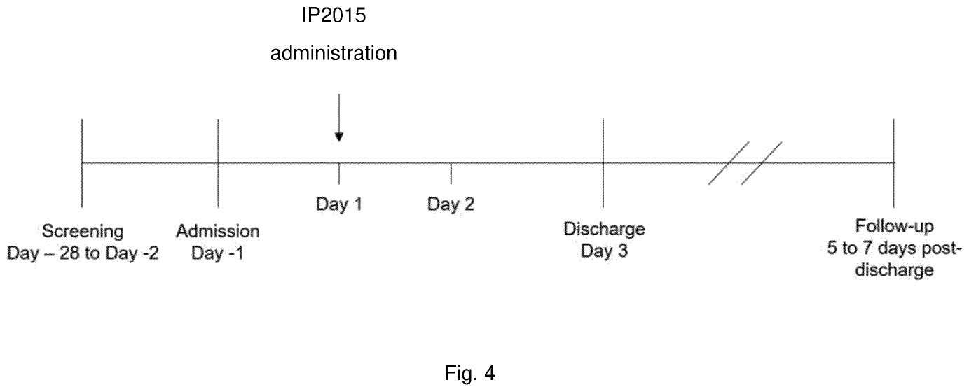

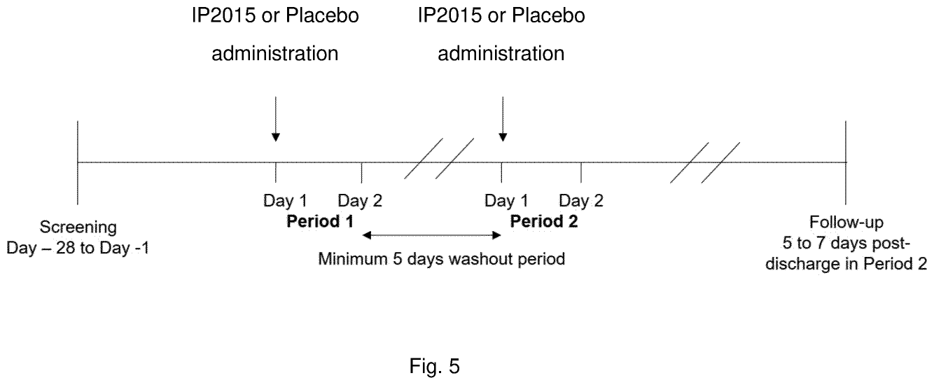

Added at basal tension, IP2015 concentration-dependently induced relaxation in corpus cavernosum strips, an effect that was unaltered in the presence of clozapine but converted to small contractions induced by IP2015 in the presence of the dopamine D1 receptor antagonist, SCH23390 (FIG. 3A). IP2015 relaxation was observed in preparations with endothelium but not in preparations without endothelium (FIG. 3B). Incubation with the NO synthase inhibitor, L-NOARG (10-4 M), abolished the relaxant effect of IP2015, while pretreatment with sildenafil (10-7 M) resulted in an enhanced relaxation to IP2015 (FIG. 3C). These findings suggest endothelial dopamine D1 receptors followed by NO release are involved in the IP2015 relaxation of corpus cavernosum.

### Conclusion

Intracavernosal pressure measurement in anaesthetized rats revealed that IP2015 dose-dependently increased the number and the duration of spontaneous erections. Whereas pretreatment with the dopamine D2-like receptor antagonist, clozapine, or cutting the cavernosal nerve inhibited IP2015-induced erectile responses, the phosphodiesterase type 5 inhibitor sildenafil further enhanced the IP2015 mediated increase in intracavernosal pressure. IP2015 also increased the number of erections in type 2 diabetic db/db mice. Direct intracavernosal injection of IP2015 increased penile pressure, and in corpus cavernosum strips, IP2015 induced concentration-dependent relaxations, which were enhanced by sildenafil and blunted by endothelial cell removal, a nitric oxide synthase inhibitor, NG-nitro-L-arginine, and a D1 receptor antagonist, SCH23390.

### Example 2. Determining the Safety and Tolerability of Ascending Single Doses of IP2015 in Healthy Male Subjects

The objectives of the study were to determine the safety and tolerability of ascending single doses of IP2015 in healthy male subjects and to determine the single oral dose pharmacokinetics (PK) of IP2015 in healthy male subjects.

### Materials and Methods

### Study Design

The study was a Phase I, randomised, double-blind, placebo-controlled, ascending single oral dose, safety, tolerability, pharmacokinetic (PK) and pharmacodynamics (PD) study of IP2015 in healthy male subjects.

Each subject received one oral dose of IP2015 or matched placebo. The starting dose of IP2015 was 0.01 mg in Cohort 1. The dose level was to be escalated to a maximum of 0.05 mg in Cohort 2 and a maximum of 0.2 mg in Cohort 3. The doses in the remaining five cohorts were to be determined based on the dose escalation criteria. In every cohort, no more than 2 subjects were dosed on the first dosing day (1 active; 1 placebo) such that no more than 1 subject received an active IP2015 dose for the first time at each dose level.

Subjects were required to attend the clinical research unit (CRU) for a screening visit within 28 days prior to dosing. Subjects were admitted to the CRU on Day-1 for collection of baseline safety and pharmacodynamics (PD) assessments (RigiScan® [Cohorts 3 to 8 only] and central nervous system (CNS) assessments [saccadic eye movement measurements, visual analogue scale [VAS] and assessment of prolactin levels; saccadic eye movement measurements were investigated in Cohorts 3 to 8 only]) and received a dose of IP2015 or placebo on the morning of Day 1, in the fasted state. Subjects in Cohorts 3 to 8 only self-applied a RigiScan Plus Monitor to their penis on Day −1 and again on Day 1 prior to dosing where it remained in place for a period of 10 hours on each occasion for RigiScan assessments. All subjects remained in the CRU until Day 3 (48 hours postdose) for the collection of safety assessments, PK blood and urine samples, CNS assessments and RigiScan assessments.

Subjects attended a follow-up visit 5 to 7 days after discharge from the CRU. The duration of participation for each subject was approximately 5 weeks.

The study design is presented in FIG. 1.

### Participants

The healthy subjects were heterosexual male, of any ethnic origin, were aged between 18 to 59 years (inclusive), had a BMI of 18 to 32 kg/m² (inclusive), and had a body weight of ≥50 kg. The participants had a score of 17 to 25 on the International Index of Erectile Function (IIEF-5) Questionnaire at screening. The participants were healthy as determined by a responsible physician, based on medical history, physical examination, concomitant medication, vital signs, 12-lead ECGs and clinical laboratory evaluations. All participants gave a written informed consent, which included compliance with the requirements and restrictions listed in the consent form.

### Randomisation and Blinding

In Cohorts 1 and 2, 5 subjects in total were randomly assigned to receive either IP2015 (3 subjects) or placebo (2 subjects). Of the first 2 subjects, one was administered placebo and one was administered IP2015. For all other cohorts 3-8, 8 subjects in total were randomly assigned to receive either IP2015 (6 subjects) or placebo (2 subjects). Of the first 2 subjects, one was administered placebo and one was administered IP2015. The randomisation schemes were generated by a statistician using SAS PROC Plan.

The study was conducted in a double-blinded fashion (Investigator and subject/patient blinded). The randomisation list was kept in a secure location until the end of the study.

The planned volume of either IP2015 or placebo was poured into a blinded dosing container and provided to the dosing staff in the CRU.

### Study Drug, Dose and Mode of Administration

IP2015 and matched placebo were provided as a powder in bottles for oral solution. A 5% hydroxy propyl beta cyclodextrin solution was reconstituted to form placebo, and this was used to dissolve IP2015. Reconstitution was performed by a pharmacist at the clinical site prior to dosing. Until IP2015 was dispensed to the subjects/participants, it was stored at a controlled room temperature of 15 to 25° C.

The IP2015 or matched placebo was administered once on the morning of Day 1 as an oral solution in the fasted state. The dose was taken with 240 ml of water at room temperature. Subjects/participants were fasted overnight prior to dosing until 4 hours postdose. Water was allowed ad libitum except for 1 hour before and 1 hour after dosing.

The doses used in each cohort were as follows:
Cohort 1-0.01 mg
Cohort 2-0.05 mg
Cohort 3-0.2 mg
Cohort 4-0.6 mg
Cohort 5-1.8 mg
Cohort 6-5.4 mg
Cohort 7-16.2 mg
Cohort 8-10 mg

### Evaluation

The study evaluated the clinical safety data from adverse event (AE) reporting, 12-lead electrocardiogram (ECG), cardiac telemetry, vital signs (standing and supine blood pressure (BP), heart rate (HR), oral temperature and clinical laboratory evaluations (chemistry, haematology, urinalysis) and physical examinations in healthy male subjects.

The study also evaluated plasma PK concentrations and parameters including but not limited to: area under the plasma concentration vs time curve (AUC), from time zero to the last quantifiable concentration (AUC0-t), AUC from zero to infinity (AUC0-∞), observed maximum plasma concentration (Cmax), time to reach maximum plasma concentration (tmax) and terminal elimination half-life (T½) in healthy male subjects. Blood samples for the determination of plasma concentrations of IP2015 were collected at predose, and 0.25, 0.5, 1, 1.5, 2, 3, 4, 6, 8, 12, 16, 24, 36 and 48 hours postdose.

The study further evaluated the relationship between PK data and PD data from CNS assessments and RigiScan assessments in healthy male subjects and the possible relationship between IP2015 dose and serum prolactin levels.

RigiScan assessments were taken on Day-1, and from 1 hour predose through 9 hours postdose on Day 1. CNS assessments (saccadic eye movement and Visual Analogue Scale [VAS] assessments) were carried out at predose, and 1, 2, 4 and 8 hours postdose on Day 1 and prolactin measurements were taken at 24 and 48 hours postdose.

Central nervous system assessments were conducted to determine the potential CNS effects of IP2015, and included the following:
Saccadic eye movement (sedation test; Cohorts 3 to 8 only in Part A): Saccadic eye movement was measured using a saccadometer, which is a miniaturised, portable device for recording saccadic responses to visual stimuli. Eye movements were measured non-invasively using infra-red reflection, and miniature lasers mounted on the transducer projected small stimuli in front of the subject. A run of 100 saccades were performed at each timepoint.
Visual Analogue Scale: A VAS was used to assess a series of symptoms (sleepy, hungry, dizzy, nauseated, anxious, irritable) on a scale from ‘not at all’ to ‘extremely’.
Prolactin levels: Serum prolactin levels were obtained via blood sample and analysed.

Subjects self-applied a RigiScan Plus Monitor to their penis on Day-1 and Day 1 prior to dosing where it stayed in place for 10 hours on each occasion.

The RigiScan Plus Monitor consists of two loops, one to be placed around the base of the penis and the other towards the tip. The ambulatory monitor uses tip and base penile loops that adjust by tightening slightly at discrete time intervals to measure and record penile rigidity and tumescence. Each loop contains a cable that moves freely inside a conduit. Each loop takes a measurement every 15 seconds. The loop gently tightens with a linear force of 4 ounces (114 g) and then immediately releases and the tissue rebounds to its unloaded state. The RigiScan Plus Monitor then takes a tumescence measurement. After the tumescence measurement is taken, it is compared to the previous samples. When a 6 mm increase in tumescence is detected, representing possible erectile activity, the RigiScan Plus Monitor takes a second measurement every 30 seconds. After tumescence is measured, the loops tighten a second time around the circumference of the penis with a linear force of 10 ounces (283.5 g). The RigiScan Plus Monitor takes a measurement when this force is applied to record a cross sectional response to radial compression. This is how rigidity is measured¹. Data were recorded on a monitor strapped to the thigh of the subjects/participants and downloaded onto a computer after the recording session. Subjects/participants were instructed on how to self-apply the RigiScan Plus Monitor by the Investigator and were advised to wear loose fitting clothing during the residential stay at the unit.

The PD parameters were derived from PD evaluation of RigiScan assessments and the CNS assessments. The PD parameters shown in Table 1 were derived to assess the effect of IP2015.

TABLE 1
Pharmacodynamic/Efficacy Assessment Parameters

| Assessment | Parameter |
|---|---|
| RigiScan Measurement | Session Duration |
| | Total Event Duration |
| | Total Event |
| | Event Duration (% of session) |
| | Average Event Regidity (%) |
| | Time Rigid 80-100% |
| | Time Rigid 60-79% |
| | Time Rigid 40-59% |
| | Time Rigid 20-39% |
| | Time Rigid 0-19% |
| | Baseline Circumference |
| | Average Events of Tumescence |
| | Event Tumescence % > Bline % |
| | Rigidity Activity Units (RAU) |
| | Tumescence Activity Units (TAU) |
| Visual Analogue Scale Assessment | Sleepy |
| | Hungry |
| | Dizzy |
| | Nauseated |
| | Anxious |
| | Irritable |
| Eye Movement Assessment | Latency |
| | Peak Saccadic Velocity |
| | Failed Saccades |

### Statistical Methods

Safety parameters were listed and summarised using descriptive statistics. Pharmacokinetic parameter estimates were calculated using noncompartmental methods. Pharmacokinetic data were listed for each subject/patient and summarised by descriptive statistics.

Dose proportionality was analysed for Part A with a linear regression model using the logarithm of a PK parameter as responsible variable and the logarithm of the dose as the independent variable. The linear regression model can be expressed as:

Log(yᵢ)=α+β*log doseᵢ+εᵢ

where α is the intercept, doseᵢ is the actual dose of IP2015 for the ith subject and εᵢ is the (within subject) random error in observing yᵢ.

The above model was applied to the following PK parameters; AUC0-t, AUC0-∞ and Cmax. Based on the linear regression model, the dose proportionality coefficient (slope) and its two-sided 90% confidence interval (CI) were estimated. Dose proportionality was declared if the 90% CI for the slope was completely contained in the following range 16: 1+log (0.5)/log (r), 1+log (2)/log (r), where r is the high dose/low dose.

Pharmacodynamic data were listed for each subject, along with summary statistics including arithmetic means, SD, minimum, maximum and median values, by timepoint and dose cohort.

Effects of IP2015 on PD parameters were analysed with a generalised mixed regression model using a PD parameter as responsible variable and dose cohort, time, interaction between dose cohort and time as mixed effects, baseline measurements as a covariate, and subject as a random effect. This model was applied to each PD parameter. The difference in least squares (LS) means between two dose cohorts at different timepoints together with 95% CI were derived from the generalised mixed regression model. For a continuous outcome, treatment difference was measured as the difference in LS mean; for a binary outcome, treatment difference was measured as the odds ratio. In addition, the dosages of the study drug were introduced as a continuous variable in the above generalised linear mixed model. In case of model non-convergence, some timepoints and dose cohorts could be pooled.

The relationship between plasma PK levels and PD response as measured by RigiScan data was explored using a generalised nonlinear mixed model.

### Results

### Single Dose Pharmacokinetics of IP2015

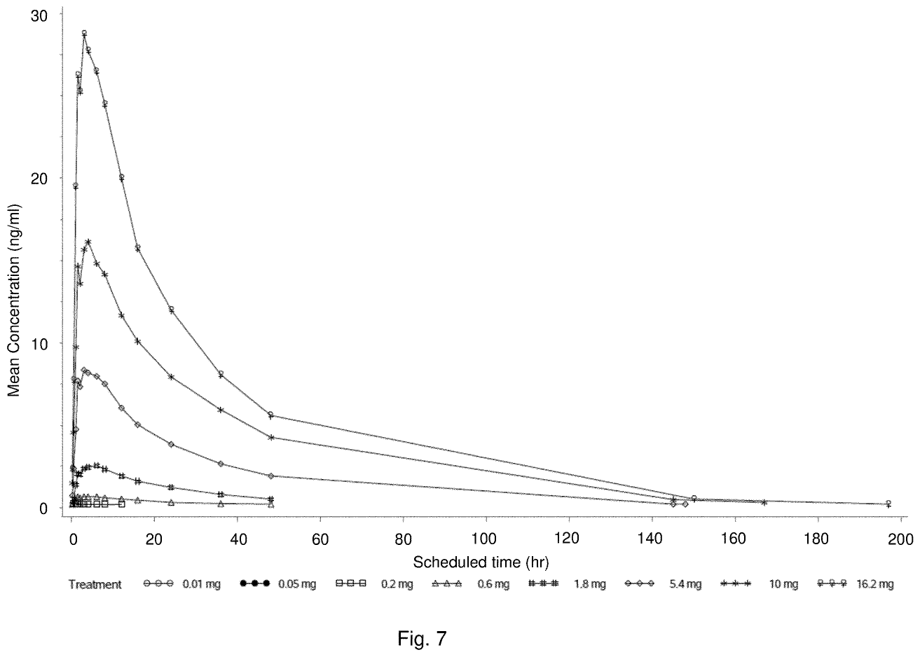

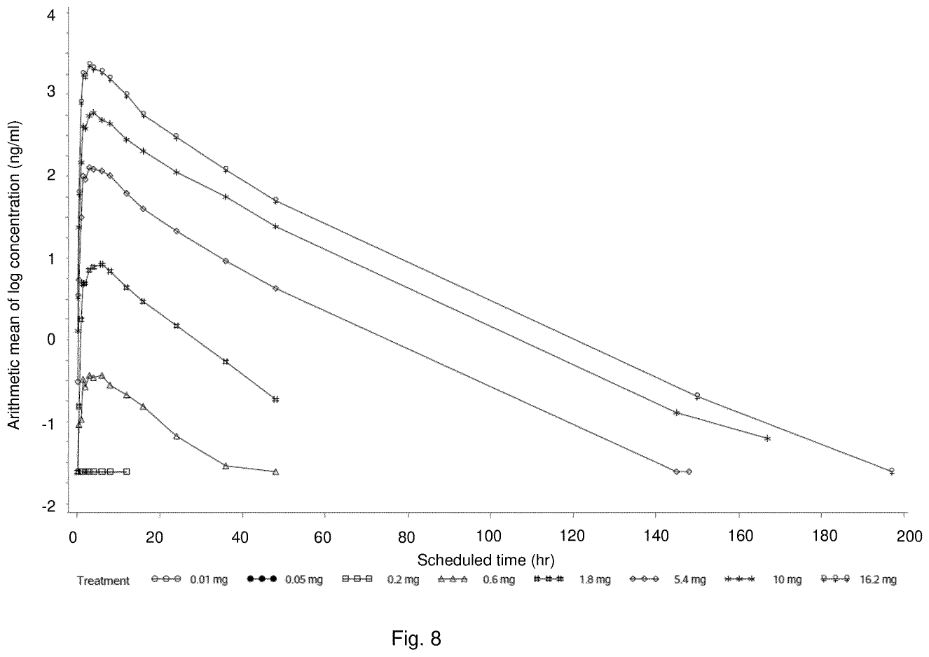

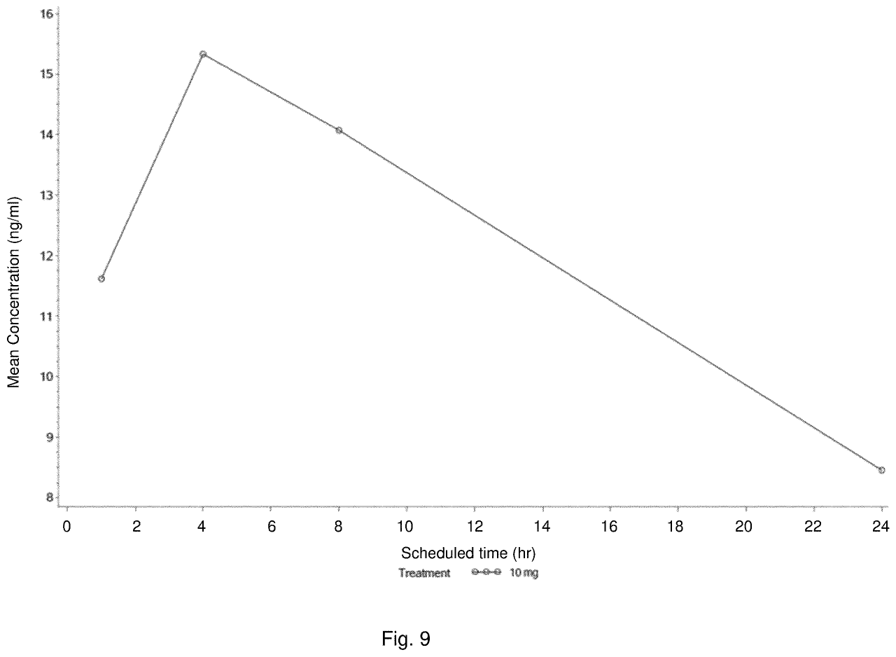

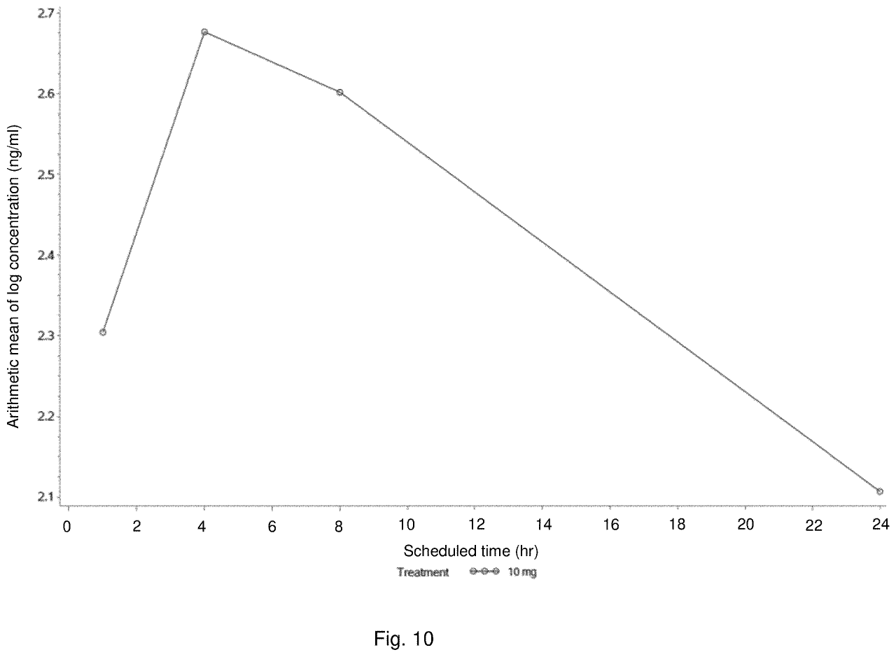

Plasma concentrations of IP2015 for each dose level in Part A are presented in FIG. 7 (linear scale) and FIG. 8 (semi-logarithmic scale).

Plasma concentrations of IP2015 were below the LLOQ for all subjects who received 0.01 mg and 0.05 mg IP2015, and for 3/7 subjects who received 0.2 mg IP2015. The plasma concentration of IP2015 versus time profiles for doses ≥0.6 mg were characterised by a relatively rapid absorption phase. Median tmax was generally similar at each dose, ranging from 2.25 to 5.00 hours postdose, and tmax ranged from 1.00 to 6.00 hours postdose across all dose levels. After reaching Cmax, plasma concentrations of IP2015 appeared to decline in a bi-phasic manner. The mean T½ of IP2015 was generally similar across the 5.4 mg to 16.2 mg doses, with mean values ranging from 23.11 to 26.30 hours. At the lower dose levels of 0.6 mg and 1.8 mg, the mean t½ was shorter (14.49 hours and 16.09 hours, respectively), probably due to the elimination phase not being fully defined at these doses as T½ could only be calculated for 1/6 subjects who received 0.6 mg IP2015 and 3/6 subjects who received 1.8 mg IP2015. Half-life could not be calculated for any subject at the 0.2 mg dose level.

Analysis of dose proportionality is presented in Table 2. The slope estimate (90% CI) from the regression analysis for Cmax was 1.1389 (1.1005 to 1.1773) for IP2015. The lower limit of the 90% CI was above unity indicating a slightly more than dose-proportional increase for Cmax over the range of 0.2 to 16.2 mg. The slope estimates (90% CI) for AUC0-∞ and AUC0-t were 1.1884 (1.0721 to 1.3048) and 1.5579 (1.4007 to 1.7151), respectively, for IP2015. The lower limit of the 90% CI was above unity for both parameters, and indicated a slightly more than dose-proportional increase in the systemic exposure based on AUC0-∞ and a greater than dose-proportional increase in the systemic exposure based on AUC0-t over the dose range 0.2 to 16.2 mg.

TABLE 2
Analysis of dose proportionality

| | | | | 90% CI | | Range | |
|---|---|---|---|---|---|---|---|
| Parameter | Effect | Estimate | P-value | Lower | Upper | Lower | Upper |
| Cmax | Intercept | 0.2406 | <0.0001 | 0.1748 | 0.3064 | — | — |
| (ng/mL) | Logdose | 1.1389 | <0.0001 | 1.1005 | 1.1773 | 0.7897 | 1.2103 |
| AUC0-t | Intercept | 2.8503 | <0.0001 | 2.5810 | 3.1179 | — | — |
| (ng/mL) | Logdose | 1.5579 | <0.0001 | 1.4007 | 1.7151 | 0.8423 | 1.1577 |
| AUC0-∞ | Intercept | 3.6152 | <0.0001 | 3.3747 | 3.8558 | — | — |
| (ng/mL) | Logdose | 1.1884 | <0.0001 | 1.0721 | 1.3048 | 0.8423 | 1.1577 |

Abbreviations: AUC0-∞—area under the plasma concentration vs time curve from zero to infinity; AUC0-t—area under the plasma concentration vs time curve from time zero to the last quantifiable concentration; CI—confidence interval; Cmax—maximum plasma concentration.

### Adverse Events:

Overall, 24 (42.1%) subjects experienced 44 treatment-emergent adverse events (TEAEs). The incidence of TEAEs was relatively low over the 0.01 mg to 10 mg dose range. In the 16.2 mg dose group, all 4 subjects reported at least one TEAE. The majority of events were mild in severity and no subjects discontinued due to a TEAE. Of the 40 subjects who received IP2015, 16 subjects experienced a total of 32 TEAEs during the study. Treatment-emergent AEs were reported for all dose groups, with the exceptions of the 0.05 mg and 0.6 mg dose groups. A total of 12 TEAEs were experienced by 8 subjects in the placebo group.

There were no significant treatment- or dose-related trends in the mean or individual subject haematology, serum biochemistry or urinalysis data during the study and no clinically significant findings in the physical examinations performed. There were no significant treatment- or dose-related trends in the mean or individual subject vital sign values over the 0.01 mg to 10 mg dose range. At the 16.2 mg dose level, increases from baseline in standing pulse rate were observed at 2, 3, 4, 6, 8, 10 and 12 hours postdose, respectively, while increases from baseline in supine pulse rate were observed at 6, 8 and 10 hours postdose, respectively. These increases from baseline were the result of 2 subjects who showed sustained tachycardia for several hours.

There were no significant treatment- or dose-related trends in the mean ECG parameters over the 0.01 mg to 10 mg dose range. At the 16.2 mg dose level, increases from baseline in heart rate were observed at 8 and 12 hours postdose, respectively.

### Conclusion

Plasma concentrations of IP2015 were below the LLOQ for all subjects who received 0.01 mg and 0.05 mg IP2015, and for 3/7 subjects who received 0.2 mg IP2015. Following single oral doses of 0.2, 0.6, 1.8, 5.4, 10 and 16.2 mg, IP2015 was relatively rapidly absorbed, with median tmax values between 2.25 and 5.00 hours. The mean T½ values ranged from 23.11 to 26.30 hours over the 5.4 mg to 16.2 mg dose range; the elimination phase could not be fully defined over the 0.2 to 1.8 mg dose range. Systemic exposure to IP2015 in plasma, based on AUC0-∞ and Cmax values, appeared to increase in a slightly more than dose-proportional manner over the 0.2 to 16.2 mg single dose range, and a greater than dose-proportional manner based on AUC0-t. The incidence of TEAEs was relatively low over the 0.01 mg to 10 mg dose range. The incidence of side effects was increased at 16.2 mg compared to the 0.01 mg to 10 mg dose range. The majority of events were mild in severity and no subjects discontinued due to a TEAE.

### Example 3. Effects of a Single Oral Dose of IP2015 on Erectile Function in Males with Erectile Dysfunction

The study investigated the effects of IP2015 on penile rigidity and tumescence in male patients with erectile dysfunction (ED) during visual stimulation, as well as the safety and tolerability of single doses of IP2015 in male patients with ED.

### Materials and Methods

### Study Design

The study was an exploratory Phase II, randomised, placebo-controlled, double-blind, crossover study, investigating the effects of a single oral dose of IP2015 on erectile function in males with ED. A dose level of 10 mg was selected. Patients were enrolled into two cohorts. Both cohorts participated in two periods (Period 1 and Period 2) and received a single dose of IP2015 and a single dose of placebo. The study design in presented in FIG. 2.

Twelve patients were enrolled into two cohorts (6 patients per cohort). Both cohorts participated in two periods (Period 1 and Period 2) and received a single dose of IP2015 and a single dose of placebo. In both cohorts, patients were required to attend the CRU for a screening visit within 28 days prior to first dosing. Patients attended the CRU on the morning on Day 1, Period 1, to undergo predose safety, PK and efficacy assessments. The efficacy assessment required the patients to self-apply the RigiScan Plus Monitor to their penis where remained in place for approximately 20 minutes at each stimulus challenge assessment.

All patients were dosed on Day 1 with either placebo or IP2015, in the fasted state. Patients remained at the CRU overnight until Day 2 (a minimum of 24 hours postdose) for completion of efficacy, safety and PK assessments, and were discharged from the CRU at the discretion of the Investigator. Following a minimum washout period of 5 days, patients returned to the CRU for Day 1 of Period 2 to receive the alternative treatment (either IP2015 or placebo). Patients attended a follow-up visit 5 to 7 days after Period 2. The duration of participation for each patient was approximately 6 weeks.

### Participants

The patients were heterosexual male, of any ethnic origin, were aged between 18 to 59 years (inclusive), had a BMI of 18 to 32 kg/m² (inclusive), and had a body weight of ≥50 kg. The patients had a score of <12 on the IIEF-5 Questionnaire at screening. Aside from ED, the patients were otherwise healthy as determined by a responsible physician, based on medical history, physical examination, concomitant medication, vital signs, 12-lead ECGs and clinical laboratory evaluations. All participants gave a written informed consent, which included compliance with the requirements and restrictions listed in the consent form.

### Randomisation and Blinding

The subjects/patients were assigned to a randomisation number in the order of recruitment. All screened subjects were identifiable throughout the study. The study was conducted in a double-blinded fashion (Investigator and subject/patient blinded). The randomisation list was kept in a secure location until the end of the study.

The planned volume of either IP2015 or placebo was poured into a blinded dosing container and provided to the dosing staff in the CRU.

### Study Drug, Dose and Mode of Administration

IP2015 and matched placebo were provided as a powder in bottles for oral solution. A 5% hydroxy propyl beta cyclodextrin solution was reconstituted to form placebo, and this was used to dissolve IP2015. Reconstitution was performed by a pharmacist at the clinical site prior to dosing. Until IP2015 was dispensed to the subjects/patients, it was stored at a controlled room temperature of 15 to 25° C.

The IP2015 at a dose of 10 mg or matched placebo was administered once on the morning of Day 1 (in Period 1 and Period 2) as an oral solution in the fasted state. The dose was taken with 240 ml of water at room temperature. Subjects/patients were fasted overnight prior to dosing until 4 hours postdose. Water was allowed ad libitum except for 1 hour before and 1 hour after dosing.

### Evaluation

The study evaluated the efficacy data from RigiScan assessments in male patients with ED and the clinical safety data from AE reporting, 12-lead ECGs, vital signs, physical examinations and clinical laboratory evaluations in male patients with ED.

Patients were assessed for efficacy using the RigiScan Plus Monitor during the stimulus challenge at predose and 1, 4 and 8-hour postdose on Day 1 of each treatment period.

The study also evaluated the plasma PK concentrations and parameters including but not limited to; AUC0-t, AUC0-∞, Cmax, tmax and T½ in male patients with ED. Blood samples for the determination of plasma concentrations of IP2015 were collected at predose, and 1, 4, 8 (post-stimulus challenge), and 24 hours postdose in each treatment period. The exploratory endpoint was the possible relationship between plasma PK levels and PD response as measured by RigiScan data.

Plasma samples for the determination of concentrations of IP2015 were analysed by using a validated liquid chromatography-tandem mass spectrometry method. The RigiScan Plus Monitor was used for efficacy during the stimulus challenge.

The design of the stimulus challenge is outlined in FIG. 3. Predose and 1, 4 and 8-hour postdose, the patients were asked to watch a 20-minute digital clip in a private room. The digital clip consisted of a 10-minute neutral digital clip followed by a 10-minute digital clip consisting of heterosexual pornography. The study staff started the digital clip and then left the room, during which there was a 1-minute delay before the neutral digital clip commenced. The neutral and pornographic digital clip were different at each assessment. RigiScan assessments were recorded through the assessment period. The RigiScan Plus Monitor was applied for approximately 20 minutes on each occasion. The patient was semi-recumbent during the assessment.

The RigiScan measurement parameters were treated as efficacy outcomes and calculated in a similar fashion as in Example 2.

### Statistical Methods

Safety parameters were listed and summarised using descriptive statistics. Pharmacokinetic parameter estimates were calculated using noncompartmental methods. Pharmacokinetic data were listed for each subject/patient and summarised by descriptive statistics.

Effects of IP2015 on PD parameters were analysed with a generalised mixed regression model using a PD parameter as responsible variable, treatment (active and placebo), timepoint, period (1 and 2), sequence (active-placebo, placebo-active), interaction between treatment and timepoint as mixed effects, baseline measurements as a covariate, and patient as a random effect. This model was applied to each PD parameter. The treatment difference between active and placebo at different timepoints together with 95% CIs was derived from the generalized mixed regression model. For a continuous outcome, treatment difference was measured as the difference in LS mean; for a binary outcome, treatment difference was measured as the odds ratio. In case of model non-convergence due to small sample size at some timepoints, those timepoints could be pooled with neighbouring timepoints.

The relationship between plasma PK levels and PD response as measured by RigiScan data was explored using a generalised nonlinear mixed model.

### Results

### Single Dose Pharmacokinetics of IP2015

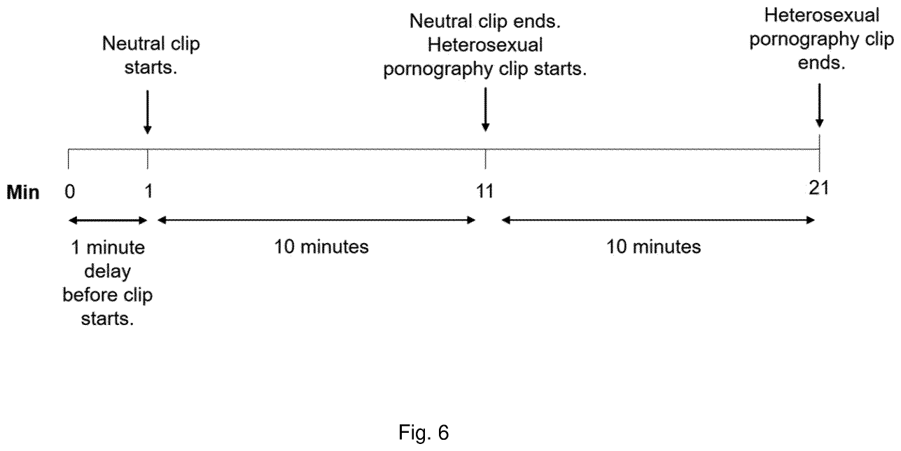

Plasma concentrations of IP2015 for the 10 mg dose level are presented in FIG. 6 (linear scale) and FIG. 7 (semi-logarithmic scale).

A limited PK profile was obtained or plasma concentrations of 10 mg IP2015 (samples taken at predose, and 1, 4, 8 and 24 hours postdose). Mean Cmax was 15.67 ng/ml and mean AUC0-t and AUC0-24 were 287.11 ng·h/mL. Median tmax occurred at 4.00 hours postdose and ranged from 1.00 to 8.53 hours postdose.

### Efficacy Evaluation

Encouraging results in favour of 10 mg IP2015 treatment compared to placebo were observed at 4 hours postdose for a number of RigiScan assessment parameters, most notably total event duration, average event rigidity, time rigid 80%-100%, average events of tumescence, RAU and TAU. The results for time rigid 80%-100% at the tip, RAU at the tip and TAU at the base were statistically significant compared to placebo (Table 3).

TABLE 3
Summary of Selected RigiScan Parameters at 4 Hours Postdose

| | | | | LS Mean | |
|---|---|---|---|---|---|
| | | Mean (SD) | | Difference | |
| | Anatomical | IP2015 | Placebo | of IP2015 − | |
| Characteristic | location | (N = 12) | (N = 12) | Placebo | 95% CI |
| Total event | | 0.09 (0.14) | 0.03 (0.04) | 0.05 | (−0.01, 0.11) |
| duration (mins) | | | | | |
| Total events | | 0.42 (0.51) | 0.42 (0.67) | 0.03 | (−0.34, 0.40) |
| Event duration | | 17.75 (27.72) | 7.33 (12.39) | 8.20 | (−5.92, 22.31) |
| (% of session) | | | | | |
| Average event | Base | 20.83 (30.61) | 15.25 (25.22) | 6.92 | (−11.73, 25.58) |
| rigidity (%) | Tip | 19.58 (29.42) | 7.75 (14.72) | 10.21 | (−4.86, 25.29) |
| Time rigid | Base | 0.04 (0.11) | 0.01 (0.02) | 0.03 | (−0.01, 0.07) |
| 80-100% | Tip | 0.04 (0.10) | 0.00 (0.00) | 0.03 | (0.00, 0.07)* |
| Baseline | Base | 7.21 (1.54) | 7.52 (1.37) | −0.28 | (−0.93, 0.37) |
| circumference | Tip | 6.82 (1.16) | 7.16 (1.18) | −0.21 | (−0.72, 0.31) |
| Average | Base | 3.49 (4.35) | 2.80 (4.20) | 1.05 | (−1.92, 4.02) |
| events of | Tip | 3.33 (4.16) | 2.55 (3.85) | 1.05 | (−1.70, 3.81) |
| tumescence | | | | | |
| RAU | Base | 3.50 (6.82) | 0.83 (1.53) | 2.33 | (−0.51, 5.17) |
| | Tip | 3.25 (6.25) | 0.33 (0.65) | 2.54 | (0.08, 4.99)* |
| TAU | Base | 2.83 (4.97) | 0.58 (1.00) | 1.95 | (0.05, 3.86)* |
| | Tip | 2.17 (3.59) | 0.42 (0.90) | 1.42 | (−0.38, 3.21) |

*Denotes a statistically significant difference compared to placebo.
Abbreviations: CI—confidence interval; LS—least squares; N—number of patients; RAU—Rigidity Activity Units; SD—standard deviation; TAU—Tumescence Activity Units

At 1 hour postdose, baseline circumference at the tip was significantly higher compared to placebo (LS mean difference 0.53 [95% CI: 0.02, 1.05]);

From the individual RAU and TAU values, 3 patients appear to be responding to treatment at 4 hours postdose (Patients 176, 177 and 191). At 4 hours postdose, the RAU values for Patients 176, 177 and 191 at the base were 21, 14 and 4, respectively, and 19, 13 and 5, respectively, at the tip. At 4 hours postdose, the TAU values for Patients 176, 177 and 191 at the base were 14, 11 and 7, respectively, and 8, 9 and 7, respectively, at the tip.

### Adverse Events:

Overall, 9 (75.0%) patients experienced 19 TEAEs. No deaths or SAEs were reported, and no subjects were discontinued due to a TEAE. All events were mild in severity.

There were no significant treatment- or dose-related trends in the mean or individual subject haematology, serum biochemistry or urinalysis data during the study and no clinically significant findings in the physical examinations performed. There were no significant treatment-related differences between IP2015 and placebo treatment for systolic blood pressure, diastolic blood pressure and oral temperature. There was an apparent treatment-related increase in both standing and supine pulse rate.

There were no significant treatment-related differences between IP2015 and placebo treatment for RR,

PR, QRS, QT and QTcF intervals. For heart rate, an increase from baseline of 14.56 bpm was observed at 8 hours postdose, while the change from baseline at the corresponding timepoint during placebo treatment was an increase of 4.78 bpm.

### Conclusion

A PK profile was obtained for plasma concentrations of 10 mg IP2015. Mean Cmax was 15.67 ng/ml and median tmax occurred at 4.00 hours postdose. Encouraging results in favour of 10 mg IP2015 treatment compared to placebo were observed at 4 hours postdose. The results for time rigid 80%-100% at the tip, RAU at the tip and TAU at the base were statistically significant compared to placebo.

### Example 4. Effects of Repeat Single Oral Doses of IP2015 on Male Subjects with Erectile Dysfunction (ED) on Ability to Develop and Maintain an Erection

The study investigated the effects of repeat single oral doses of IP2015 on male subjects with erectile dysfunction (ED) on ability to develop and maintain an erection, as well as safety and tolerability of single oral doses of IP2015, effects on penile rigidity and tumescence during visual stimulation, effects on sperm count and motility, and any possible relationship between plasma levels of IP2015, its efficacy and safety.

### Materials and Methods

### Study Design

130 subjects were divided into 3 groups upon which the study was conducted in parallel. Each subject was dosed on four occasions with either 5 mg IP2015, 10 mg IP2015 or matched placebo. In each group, subjects were randomised evenly to each of the three study treatments and received the same treatment at each visit.

The study lasted approximately 8 weeks and consisted of the following:
A screening visit (up to 21 days prior to the baseline visit)
A baseline visit (Day −7).
Outpatient visits on Week 1 (Day 1), Week 2, Week 3 and Week 4
A follow-up visit a minimum of 7 to 10 days after their final dose.

On Day-7, and at the Week 1 (Day 1), Week 2, Week 3, Week 4 and Follow-up visits, all groups were asked to complete the IIEF-15 questionnaire. Group 2 was asked to complete a Visual Stimulation Assessment using a RigiScan Plus Monitor on Day-7, Week 1 (Day 1) and Week 4. Group 2 was also required to provide blood samples for pharmacokinetic assessment on Week 1 (Day 1) and Week 4. Group 3 was required to provide semen samples on Day-7 and Week 4.

### Participants

The patients were otherwise healthy male subjects with ED as determined from an IIEF-5 score of ≤16, with a body mass index of 18 to 35 kg/m² (inclusive), of any ethnic origin. Subjects were aged between 18 to 59 years, inclusive.

### Weight at Screening:

| | 5 mg IP2015 | 10 mg IP2015 | Placebo | Overall |
|---|---|---|---|---|
| N | 42 | 43 | 45 | 130 |
| Mean | 87.42 | 86.04 | 88.06 | 87.18 |
| SD | 13.050 | 15.122 | 13.968 | 13.994 |
| Minimum | 62.6 | 61.0 | 58.8 | 58.8 |
| Median | 87.30 | 84.90 | 86.80 | 86.70 |
| Maximum | 114.8 | 126.0 | 115.0 | 126.0 |

BMI at Screening (kg/m²):

| | 5 mg IP2015 | 10 mg IP2015 | Placebo | Overall |
|---|---|---|---|---|
| N | 42 | 43 | 45 | 130 |
| Mean | 27.63 | 27.97 | 27.25 | 27.61 |
| SD | 3.556 | 3.671 | 3.420 | 3.533 |
| Minimum | 20.5 | 19.7 | 21.0 | 19.7 |
| Median | 28.25 | 27.10 | 27.00 | 27.80 |
| Maximum | 35.1 | 35.0 | 33.9 | 35.1 |

### Dose and Mode of Administration

There were three potential study treatments of 5 mg IP2015, 10 mg IP2015 or matched placebo. In each group, subjects were randomised evenly to each of the three study treatments and received the same treatment on each visit. IP2015 or matched placebo was administered once in the morning of Week 1 (Day 1), Week 2, Week 3, and Week 4 as an oral solution in the fasted state. The dose was taken with 240 ml of water at room temperature. Subjects had fasted 2 hours prior to dosing until 4 hours postdose. Water was allowed ad libitum except for 1 hour before and 1 hour after dosing.

### Evaluation

Efficacy was assessed through the International Index of Erectile Function (IIEF)-15 questionnaire. The change from baseline in responses to questions of the IIEF-15 questionnaire, including questions regarding erectile function, orgasmic function, sexual desire, intercourse satisfaction and overall satisfaction domain scores, was determined at different time points of the study. RigiScan assessments during the stimulus assessment, and sperm count and motility by semen sample collection was used for efficacy evaluation. Safety was assessed through AE reporting, 12-lead ECG, vital signs, physical examinations and clinical laboratory evaluations. Pharmacokinetics was assessed by blood sampling.

### Results

### Efficacy of IP2015

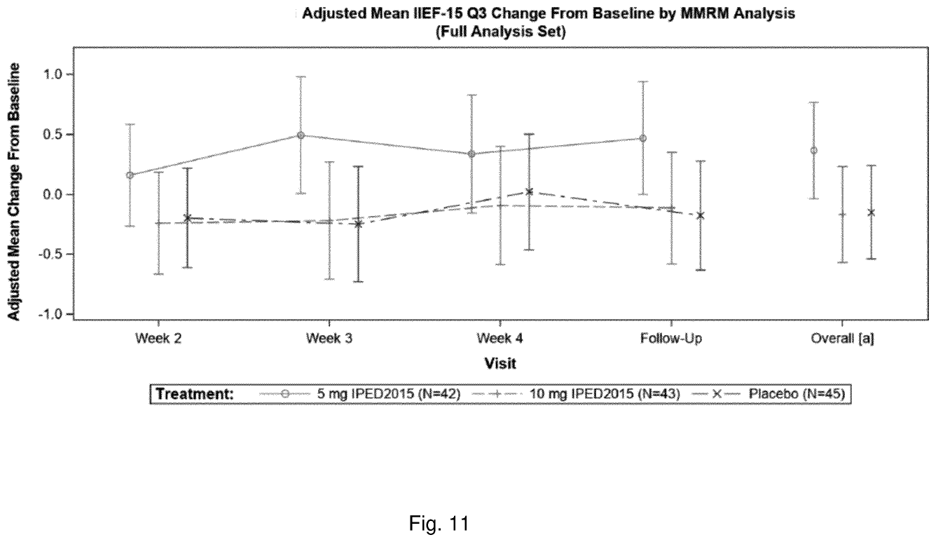

The results for Q3 in the IIEF-15: “When you attempted intercourse, how often were you able to penetrate (enter) your partner during the last week?” are shown in FIG. 11. The results for Q3 were significant for treatment with 5 mg IP2015 at week 3 versus placebo (p=0.034) and versus baseline (p=0.046). In the overall score for Q3, there was also a tendency to difference versus placebo (p=0.07) and versus baseline (p=0.07).

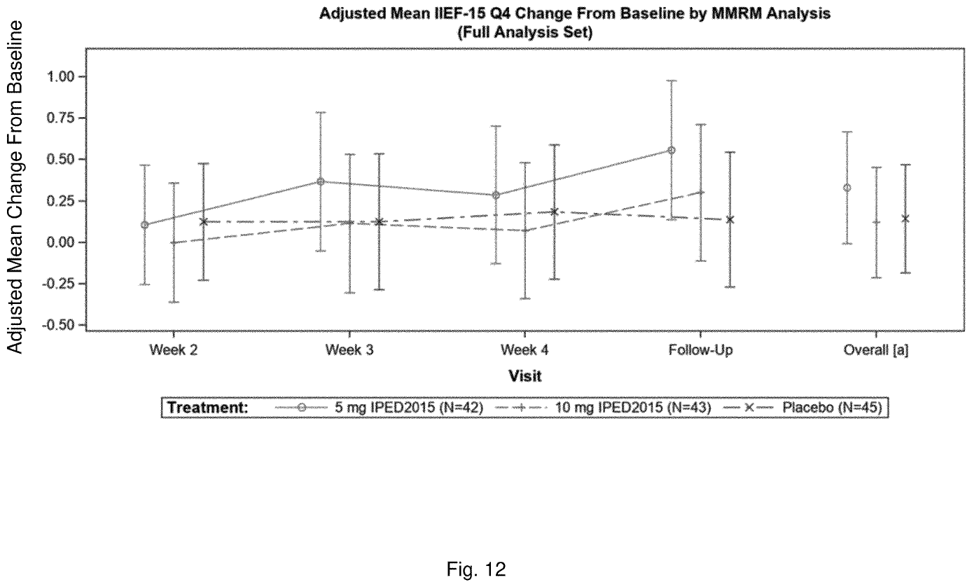

The results for Q4 in the IIEF-15: “During sexual intercourse, how often were you able to maintain your erection after you had penetrated (entered) your partner?” are shown in FIG. 12. The results for Q4 show that there was a tendency to difference versus baseline (p=0.056) for the 5 mg in the overall score, though there was no difference compared to placebo (p=0.44).

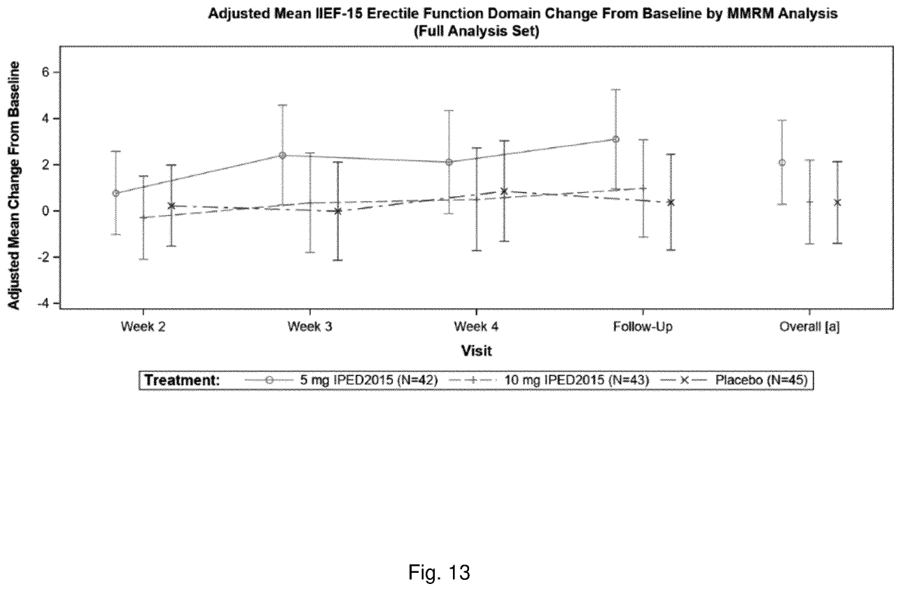

The overall response to questions of IIEF-15 is shown in FIG. 13, the results were significant for treatment with 5 mg IP2015 versus baseline at follow-up (p=0.0046) and tendency versus placebo (p=0.07) and the same was the case for the overall score for 5 mg versus baseline (p=0.0032) and tendency versus placebo (p=0.10).

In FIGS. 11, 12 and 13, the results show the change from baseline for patients treated with four doses of placebo (n=45), 5 mg (n=42), and 10 mg (n=43) IP2015 dosed in parallel at days 1, 8, 15, and 22. The results are means±SE. The statistical differences were performed with Mixed Model Repeated Measures (MMRM).

### Adverse Events

Treatment-emergent adverse effects for the low-dose (5 mg) IP2015 were comparable to the placebo group. The TEAEs were dose-dependent, mild and moderate and slightly increased in the high dose compared to the low dose of IP2015. No severe side effects were observed at any of the doses of IP2015 (Table 4).

The results for semen analysis was complete and the treatment did not have negative effects on sperm count, motility, or morphology.

TABLE 4
Treatment-emergent adverse events

| | IP2015 5 mg, | IP2015 10 mg, | Placebo, |
|---|---|---|---|
| TEAE | N = 42 | N = 43 | N = 45 |
| Any TEAE | 32 (76.2%), 61 | 34 (79.1%), 141 | 31 (68.9%), 63 |
| Related* | 10 (23.8%), 19 | 23 (53.5%), 84 | 8 (17.8%), 11 |
| Mild | 29 (69.0%), 56 | 33 (76.7%), 132 | 27 (60.0%), 57 |
| Moderate | 3 (7.1%), 5 | 7 (16.3%), 9 | 6 (13.3%), 6 |
| Severe | 0 | 0 | 0 |
| Headache | 11 (26.2%), 15 | 16 (37.2%), 27 | 14 (31.1%), 21 |
| Dizziness | 3 (7.1%), 3 | 12 (27.9%), 15 | 2 (4.4%), 2 |
| Nausea | 1 (2.4%), 1 | 8 (18.6%), 15 | 1 (2.2%), 1 |

### Conclusion

There was a significant effect of the 5 mg dose of IP2015 (pudafensine) on the Q3 in the IIEF-15 score compared to baseline and placebo.

There were tendencies regarding the overall IIEF-15 score showing a clinically significant score change of 2, which is significant for a low dose of IP2015 versus baseline and p=0.10 versus placebo.

The treatment did not have negative effects on sperm count, motility, or morphology. There was a linear relation of plasma concentrations to dose of IP2015.

Treatment-emergent adverse effects for the 5 mg dose of IP2015 were comparable to the placebo group. The TEAEs were dose-dependent, only mild and moderate effects were observed. No severe TEAEs were present.

Overall these results, show that 5 mg doses that IP2015 are effective in treating erectile dysfunction and are safely tolerated.

### REFERENCES

WO 97/30997
GOTOP Medical. RigiScan PLUS Rigidity Assessment System [internet] 2014. Available from: http://www.gotopmedical.com/rigiscan®-plus.html.

1. A compound of formula (I), 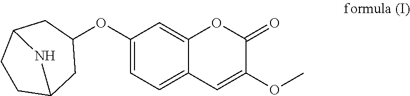 [SMILES: COc1cc2ccc(OC3CC4CCC(C3)N4)cc2oc1=O]
or a pharmaceutically acceptable salt thereof, for use in the treatment, prevention, reduction, or alleviation of erectile dysfunction in a subject, wherein the compound is administered in an amount from about 0.1 mg to about 10 mg per individual dose.

2. The compound for use according to any one of claims 1 and 2, wherein the compound has the structure of formula (Ia); 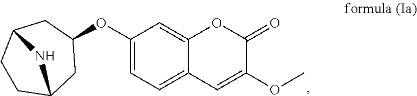 [SMILES: COc1cc2ccc(O[C@@H]3C[C@H]4CC[C@@H](C3)N4)cc2oc1=O]
or a pharmaceutically acceptable salt thereof.

3. The compound for use according to any one of the preceding claims, wherein the compound is exo-7-[(8-azabicyclo[3.2.1]octan-3-yl)oxy]-3-methoxy-chromen-2-one or a pharmaceutically acceptable salt thereof.

4. The compound for use according to any one of the preceding claims wherein the compound is exo-7-[(8-azabicyclo[3.2.1]octan-3-yl)oxy]-3-methoxy-chromen-2-one hydrochloride.

5. The compound for use according to any one of the preceding claims, wherein the compound of formula I is administered in an amount per individual dose from about 0.5 mg to about 10 mg.

6. The compound for use according to any one of the preceding claims, wherein the compound of formula I is administered in an amount per individual dose from about 0.1 mg to 20 mg per individual dose, such as about 0.1 mg, such as about 0.2 mg, 0.3 mg, 0.4 mg, 0.5 mg, 0.6 mg, 0.7 mg, 0.8 mg, 0.9 mg, 1 mg, 1.5 mg, 2 mg, 2.5 mg, 3 mg, 3.5 mg, 4 mg, 4.5 mg, 5 mg, 6 mg, 7 mg, 8 mg, 9 mg, or 10 mg per individual dose.

7. The compound for use according to any one of the preceding claims, wherein the compound of formula I is administered to the subject at about 5 mg per individual dose.

8. The compound for use according to any one of the preceding claims, wherein the compound of formula I is administered to the subject at 5 mg per individual dose.

9. The compound for use according to any one of the preceding claims wherein the compound is administered once daily.

10. The compound for use according to any one of the preceding claims, wherein said compound is administered more than once a day, such as 2 times a day, such as 3 times a day, such as 4 times a day.

11. The compound for use according to any one of the preceding claims wherein the compound is administered once a week or twice a week.

12. The compound for use according to any one of the preceding claims wherein the total daily dose of said compound is from about 1 and 10 mg.

13. The compound for use according to any one of the preceding claims, wherein said compound is administered orally in an amount per individual dose from about 0.035 mg/kg to 0.135 mg/kg, such as from 0.040 mg/kg to 0.110 mg/kg, such as from 0.042 mg/kg to 0.100 mg/kg, such as from 0.0450 mg/kg to 0.100 mg/kg, such as from 0.045 mg/kg to 0.091 mg/kg, such as from 0.050 mg/kg to 0.091 mg/kg.

14. The compound for use according to any one of the preceding claims, wherein said compound is administered orally in an amount per individual dose from about from 0.075 mg/kg to 0.250 mg/kg, such as from 0.080 mg/kg to 0.222 mg/kg, such as from 0.083 mg/kg to 0.200 mg/kg, such as from 0.091 mg/kg to 0.185 mg/kg, such as from 0.100 mg/kg to 0.185 mg/kg, such as from 0.050 mg/kg to 0.185 mg/kg.

15. The compound for use according to any one of the preceding claims, wherein said compound is administered to the subject at 5 mg per individual dose, wherein the subject was a weight from about 60 kg to about 115 kg.

16. The compound for use according to any one of the preceding claims, wherein the erectile dysfunction is:
(i) associated with, or due to, a medical condition, such as injury, the effects of surgery or radiation treatment; and/or
(ii) associated with, or due to, psychological or behavioural factors, including mental disorders; and/or
(iii) associated with, or due to, use of medication or use of a psychoactive substance.

17. The compound for use according to any one of the preceding claims, wherein the erectile dysfunction is organic erectile dysfunction.

18. The compound for use according to any one of the preceding claims, wherein the erectile dysfunction is lifelong erectile dysfunction or acquired erectile dysfunction.

19. The compound for use according to any one of the preceding claims, wherein the erectile dysfunction is generalised erectile dysfunction or situational erectile dysfunction.

20. The compound for use according to any one of the preceding claims, wherein the erectile dysfunction is comorbid in the subject with one or more condition(s) selected from the group consisting of:
(i) diabetes,
(ii) metabolic syndrome
(iii) a vascular disease, such as a cardiovascular disease, and
(iv) a neurological condition.

21. The compound for use according to any one of claims 1 to 20, wherein the erectile dysfunction is associated with, or due to, metabolic syndrome.

22. The compound for use according to any one of claims 1 to 20, wherein the erectile dysfunction is associated with, or due to, diabetes.

23. The compound for use according to any one of claims 1 to 20, wherein the erectile dysfunction is associated with, or due to, a vascular disease, such as a cardiovascular disease.

24. The compound for use according to any one of claims 1 to 20, wherein the erectile dysfunction is associated with, or due to, a neurological condition.

25. The compound for use according to any one of claims 1 to 20, wherein the erectile dysfunction is treatment-emergent erectile dysfunction associated with or due to the use of medication.

26. The compound for use according to claim 25, or a pharmaceutically acceptable salt thereof, wherein the treatment-emergent erectile dysfunction is an adverse effect originating from treatment of a medicament.

27. The compound for use according to any one of claim 25 or 26, wherein the medicament is selected from the group consisting of antidepressants, NSAIDs, finasteride, antiepileptics and neuroleptics.

28. The compound for use according to any one of claim 25 or 26, wherein the erectile dysfunction is treatment-emergent erectile dysfunction caused by the treatment by and antidepressant medicament.

29. The compound for use according to any one of the preceding claims, wherein the subject is administered with a further therapeutic agent effective for the treatment of erectile dysfunction.

30. The compound for use according to claim 29, wherein the subject is administered with a further therapeutic agent effective for the treatment of erectile dysfunction selected from the group consisting of phosphodiestearase-5 (PDE5) inhibitors, such as sildenafil, tadalafil, vardenafil or avanafil; alprostadil; testosterone.

31. The compound for use according to claim 29, wherein the treatment is combined with cell therapy.

32. The compound for use according to claim 29, wherein the treatment is combined with extracorporal shock therapy.

33. The compound for use according to any one of the preceding claims, wherein the subject is under another treatment for erectile dysfunction.

34. The compound for use according to claim 33, wherein the subject is under treatment with one or more phosphodiestearase-5 (PDE5) inhibitor(s), such as sildenafil, tadalafil, vardenafil or avanafil.

35. The compound for use according to any one of the preceding claims, wherein the subject is a non-responder of treatment for erectile dysfunction with one or more PDE5 inhibitors.

36. The compound for use according to any one of the preceding claims, wherein the compound is able to produce a central effect initiating erection and/or a peripheral effect potentiating erection.

37. The compound for use according to any one of the preceding claims, wherein the compound is able to initiate and/or potentiate erection through smooth muscle relaxation.

38. The compound for use according to any one of the preceding claims, wherein the compound is able to initiate erection by increasing central dopamine and/or a peripheral effect potentiating erection through nitric oxide release.

39. The compound for use according to any one of the preceding claims, wherein the compound is able to increase the number of erectile events, the duration of erectile events, penile tumescence and/or the penile rigidity.

40. The compound for use according to any one of the preceding claims, wherein the compound is able to increase the number of erectile events, the duration of erectile events, penile tumescence and/or the penile rigidity in the subject during sexual stimulation.

41. The compound according to any one of the preceding claims, wherein the subject is a mammal.

42. The compound according to any one of the preceding claims, wherein the mammal is a human.

43. The compound according to any one of the preceding claims, wherein the human is male.

44. The compound for use according to any one of the preceding claims, wherein the subject is male.

45. The compound for use according to any one of the preceding claims, wherein the subject is an adult male.

46. The compound for use according to any one of the preceding claims, wherein the subject is a male over the age of 20.

47. The compound for use according to any one of the preceding claims, wherein the subject has a weight from about 55 kg to about 130 kg, such as 60 kg to about 115 kg.

48. The compound for use according to any one of the preceding claims, wherein the subject has a body-mass index (BMI) from about 19.5 kg/m² to about 35.5 kg/m².

49. The compound for use according to any one of the preceding claims, wherein the subject prior to treatment had an international index of Erectile Function 5 (IIEF-5) of less than 17, such as between 12 and 16, such as between 8 and 11, such as between 5 and 7.

50. The compound for use according to any one of the preceding claims, wherein the subject prior to treatment had an international index of Erectile Function 5 (IIEF-5) of less than 12.

51. The compound for use according to any one of the preceding claims, wherein the compound is able to increase the International Index of Erectile Function in the subject.

52. The compound for use according to any one of the preceding claims, wherein the administration of the compound is an oral administration.

53. The compound for use according to any one of the preceding claims, wherein the administration of the compound is parenteral administration, such as cutaneous, mucosal, subcutaneous, intramuscular, intraperitoneal, intravenous or intra-arterial injection.

54. The compound for use according to any one of the preceding claims, wherein the compound is formulated in a pharmaceutical composition further comprising a pharmaceutically acceptable diluent, carrier and/or excipient.

55. The compound for use according to any one of the preceding claims, wherein the compound is formulated as a solid dosage form, such as a tablet, a capsule, a pill, granules or a powder.

56. A solid dosage form comprising a compound of formula I, 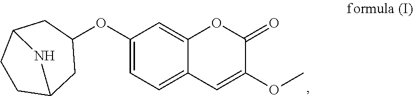 [SMILES: COc1cc2ccc(OC3CC4CCC(C3)N4)cc2oc1=O]
or a pharmaceutically acceptable salt thereof, wherein the compound is present in an amount from about 0.5 mg to about 10 mg.

57. A composition comprising a compound of formula (I), 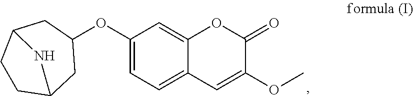 [SMILES: COc1cc2ccc(OC3CC4CCC(C3)N4)cc2oc1=O]
and one or more PDE5 inhibitor(s).

58. The composition according to claim 57, wherein the one or more PDE5 inhibitor(s) are selected from the group consisting of: sildenafil, tadalafil, vardenafil and avanafil.

59. The composition according to any one of claims 57 to 58, wherein the compound of formula (I) is present in an amount from about 0.5 mg to about 10 mg.

60. Use of a compound of formula (I), 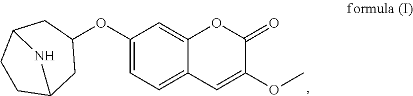 [SMILES: COc1cc2ccc(OC3CC4CCC(C3)N4)cc2oc1=O]
for the manufacture of a medicament for the treatment, prevention, reduction or alleviation of erectile dysfunction in a subject in need thereof, wherein said compound is administered in a dose from about 0.001 to about 1 mg/kg mg.

61. A method of treatment, prevention, reduction or alleviation of erectile dysfunction in a subject in need thereof, the method comprising administering to the subject in need an amount from about 0.001 to about 1 mg/kg of a compound of formula (I), 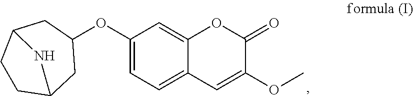 [SMILES: COc1cc2ccc(OC3CC4CCC(C3)N4)cc2oc1=O]
or a pharmaceutically acceptable salt thereof.

62. A method of increasing frequency of erectile responses in a subject, said method comprising administering to the subject an amount from about 0.001 mg/kg to about 1 mg/kg of a compound of formula (I) or a pharmaceutically acceptable salt thereof.

63. A method of increasing duration of erectile responses in a subject, said method comprising administering to the subject an amount from about 0.001 mg/kg to about 1 mg/kg of a compound of formula (I) or a pharmaceutically acceptable salt thereof.

64. A method of increasing the magnitude of erectile responses in a subject said method comprising administering to the subject an amount from about 0.001 mg/kg to about 1 mg/kg of a compound of formula (I) or a pharmaceutically acceptable salt thereof.

65. A method of achieving a mean plasma concentration of a compound of formula (I) from about 1 ng/ml to 50 ng/mL in a subject said method comprising administering to the subject an amount from about 0.001 mg/kg to about 1 mg/kg of the compound of formula (I) or a pharmaceutically acceptable salt thereof.

66. The method according to claim 65, wherein the subject is administered with an amount from about 0.5 mg to about 10 mg of the compound of formula (I).
